SEQUENCE LISTING (174 sequences)

SEQ ID NO: 1 - protein, 598 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 2 - protein, 42 aa, synthetic construct
MKKRTYCTYCPSKIRRKANASCKKCKKVICREHNIDMCQSCF

SEQ ID NO: 3 - protein, 548 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTS

SEQ ID NO: 4 - protein, 556 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPV

SEQ ID NO: 5 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 6 - protein, 109 aa, synthetic construct
SFVPNKEQTRTVLIFCFHLKKTAAESHRMLVEAFGEQVPTVKTCERWFQRFKSGDFDVDDKEHGKPPKRYEDAELQALLDEDDAQTQKQLAEQLEVSQQAVSNRLREMG

SEQ ID NO: 7 - protein, 669 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSGGGGSSFVPNKEQTRTVLIFCFHLKKTAAESHRMLVEAFGEQVPTVKTCERWFQRFKSGDFDVDDKEHGKPPKRYEDAELQALLDEDDAQTQKQLAEQLEVSQQAVSNRLREMG

SEQ ID NO: 8 - protein, 56 aa, synthetic construct
SFVPNKEQTRTVLIFCFHLKKTAAESHRMLVEAFGEQVPTVKTCERWFQRFKSGDF

SEQ ID NO: 9 - protein, 616 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSGGGGSSFVPNKEQTRTVLIFCFHLKKTAAESHRMLVEAFGEQVPTVKTCERWFQRFKSGDF

SEQ ID NO: 10 - protein, 677 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVGGGGSSFVPNKEQTRTVLIFCFHLKKTAAESHRMLVEAFGEQVPTVKTCERWFQRFKSGDFDVDDKEHGKPPKRYEDAELQALLDEDDAQTQKQLAEQLEVSQQAVSNRLREMG

SEQ ID NO: 11 - protein, 624 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVGGGGSSFVPNKEQTRTVLIFCFHLKKTAAESHRMLVEAFGEQVPTVKTCERWFQRFKSGDF

SEQ ID NO: 12 - DNA, 35 bp, synthetic construct
  source 1..35: mol_type = other DNA
ccctagaaagatagtctgcgtaaaattgacgcatg

SEQ ID NO: 13 - DNA, 23 bp, synthetic construct
  source 1..23: mol_type = other DNA
tgtacaagtatgaaatgtcgttt

SEQ ID NO: 14 - DNA, 22 bp, synthetic construct
  source 1..22: mol_type = other DNA
tgtacaagtatgaaatgtcgtt

SEQ ID NO: 15 - DNA, 39 bp, synthetic construct
  source 1..39: mol_type = other DNA
ccctagaaagatagtcaaacgacatttcatacttgtaca

SEQ ID NO: 16 - DNA, 38 bp, synthetic construct
  source 1..38: mol_type = other DNA
ccctagaaagatagtcaacgacatttcatacttgtaca

SEQ ID NO: 17 - DNA, 40 bp, synthetic construct
  source 1..40: mol_type = other DNA
ccctagaaagatagtctaaacgacatttcatacttgtaca

SEQ ID NO: 18 - DNA, 41 bp, synthetic construct
  source 1..41: mol_type = other DNA
ccctagaaagatagtctgaaacgacatttcatacttgtaca

SEQ ID NO: 19 - DNA, 39 bp, synthetic construct
  source 1..39: mol_type = other DNA
ccctagaaagataatcaaacgacatttcatacttgtaca

SEQ ID NO: 20 - DNA, 38 bp, synthetic construct
  source 1..38: mol_type = other DNA
ccctagaaagataatcaacgacatttcatacttgtaca

SEQ ID NO: 21 - DNA, 40 bp, synthetic construct
  source 1..40: mol_type = other DNA
ccctagaaagataatcaaaacgacatttcatacttgtaca

SEQ ID NO: 22 - DNA, 41 bp, synthetic construct
  source 1..41: mol_type = other DNA
ccctagaaagataatcataaacgacatttcatacttgtaca

SEQ ID NO: 23 - protein, 99 aa, synthetic construct
GGGGSERPYACPVESCDRRFSRSDELTRHIRIHTGQKPFQCRICMRNFSRSDHLTTHIRTHTGEKPFACDICGRKFARSDERKRHTKIHLRQKDGGGGS

SEQ ID NO: 24 - protein, 620 aa, synthetic construct
MAPKKKRKVGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSGGGGSSFVPNKEQTRTVLIFCFHLKKTAAESHRMLVEAFGEQVPTVKTCERWFQRFKSGDF

SEQ ID NO: 25 - protein, 673 aa, synthetic construct
MAPKKKRKVGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSGGGGSSFVPNKEQTRTVLIFCFHLKKTAAESHRMLVEAFGEQVPTVKTCERWFQRFKSGDFDVDDKEHGKPPKRYEDAELQALLDEDDAQTQKQLAEQLEVSQQAVSNRLREMG

SEQ ID NO: 26 - protein, 628 aa, synthetic construct
MAPKKKRKVGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVGGGGSSFVPNKEQTRTVLIFCFHLKKTAAESHRMLVEAFGEQVPTVKTCERWFQRFKSGDF

SEQ ID NO: 27 - protein, 681 aa, synthetic construct
MAPKKKRKVGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVGGGGSSFVPNKEQTRTVLIFCFHLKKTAAESHRMLVEAFGEQVPTVKTCERWFQRFKSGDFDVDDKEHGKPPKRYEDAELQALLDEDDAQTQKQLAEQLEVSQQAVSNRLREMG

SEQ ID NO: 28 - skipped

SEQ ID NO: 29 - protein, 278 aa, synthetic construct
SIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPTHEGDQRRASSRKRSRSDRAVTGPSAQQSFEVRAPEQRDALHLPLSWRVKRPRTSIGGGLPDPGTPTAADLAASSTVMREQDEDPFAGAADDFPAFNEEELAWLMELLPQ

SEQ ID NO: 30 - protein, 136 aa, synthetic construct
VDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 31 - protein, 63 aa, synthetic construct
SIVAQLSRPDPALAALTNDHLVALACLGGRPALDAVKKGLPHAPALIKRTNRRIPERTSHRVA

SEQ ID NO: 32 - protein, 136 aa, synthetic construct
VDLRTLGYSQQQQEKIKPKVRSTVAQHHEALVGHGFTHAHIVALSQHPAALGTVAVKYQDMIAALPEATHEAIVGVGKSRSGARALEALLTVAGELRGPPLQLDTGQLLKIAKRGGVTAVEAVHAWRNALTGAPLN

SEQ ID NO: 33 - protein, 135 aa, synthetic construct
VDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLN

SEQ ID NO: 34 - protein, 34 aa, synthetic construct
  VARIANT 11..13: note = X is any amino acid
LTPDQVVAIAXXXGGKQALETVQRLLPVLCQDHG

SEQ ID NO: 35 - protein, 34 aa, synthetic construct
  VARIANT 11..13
LTPEQVVAIAXXXGGKQALETVQRLLPVLCQAHG

SEQ ID NO: 36 - protein, 34 aa, synthetic construct
  VARIANT 11..13
LTPDQVVAIAXXXGGKQALETVQRLLPVLCQAHG

SEQ ID NO: 37 - protein, 34 aa, synthetic construct
  VARIANT 11..13
LTPAQVVAIAXXXGGKQALETVQRLLPVLCQDHG

SEQ ID NO: 38 - protein, 20 aa, synthetic construct
  VARIANT 11..13
LTPEQVVAIAXXXGGRPALE

SEQ ID NO: 39 - protein, 7 aa, synthetic construct
PKKKRKV

SEQ ID NO: 40 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 41 - protein, 1082 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSGGGGSSFVPNKEQTRTVLIFCFHLKKTAAESHRMLVEAFGEQVPTVKTCERWFQRFKSGDF

SEQ ID NO: 42 - protein, 1090 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVGGGGSSFVPNKEQTRTVLIFCFHLKKTAAESHRMLVEAFGEQVPTVKTCERWFQRFKSGDF

SEQ ID NO: 43 - protein, 1135 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSGGGGSSFVPNKEQTRTVLIFCFHLKKTAAESHRMLVEAFGEQVPTVKTCERWFQRFKSGDFDVDDKEHGKPPKRYEDAELQALLDEDDAQTQKQLAEQLEVSQQAVSNRLREMG

SEQ ID NO: 44 - protein, 1143 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVGGGGSSFVPNKEQTRTVLIFCFHLKKTAAESHRMLVEAFGEQVPTVKTCERWFQRFKSGDFDVDDKEHGKPPKRYEDAELQALLDEDDAQTQKQLAEQLEVSQQAVSNRLREMG

SEQ ID NO: 45 - DNA, 1264 bp, synthetic construct
  source 1..1264: mol_type = other DNA
agctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 46 - DNA, 684 bp, synthetic construct
  source 1..684: mol_type = other DNA
atgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccattaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatga

SEQ ID NO: 47 - DNA, 225 bp, synthetic construct
  source 1..225: mol_type = other DNA
gagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagacc

SEQ ID NO: 48 - DNA, 236 bp, synthetic construct
  source 1..236: mol_type = other DNA
atgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccattaa

SEQ ID NO: 49 - DNA, 452 bp, synthetic construct
  source 1..452: mol_type = other DNA
ttaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatga

SEQ ID NO: 50 - DNA, 353 bp, synthetic construct
  source 1..353: mol_type = other DNA
ttaaccctagaaagatagtcaaacgacatttcatacttgtacagacgcatgcattcttgaaatattgctctctctttctaaatagcgcgaatccgtcgctgtgcatttaggacatctcagtcgcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaaatgtacaagtatgaaatgtcgtttgattatctttctagggttaa

SEQ ID NO: 51 - DNA, 351 bp, synthetic construct
  source 1..351: mol_type = other DNA
ttaaccctagaaagatagtcaacgacatttcatacttgtacagacgcatgcattcttgaaatattgctctctctttctaaatagcgcgaatccgtcgctgtgcatttaggacatctcagtcgcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaaatgtacaagtatgaaatgtcgttgattatctttctagggttaa

SEQ ID NO: 52 - DNA, 355 bp, synthetic construct
  source 1..355: mol_type = other DNA
ttaaccctagaaagatagtctaaacgacatttcatacttgtacagacgcatgcattcttgaaatattgctctctctttctaaatagcgcgaatccgtcgctgtgcatttaggacatctcagtcgcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaaatgtacaagtatgaaatgtcgttttgattatctttctagggttaa

SEQ ID NO: 53 - DNA, 357 bp, synthetic construct
  source 1..357: mol_type = other DNA
ttaaccctagaaagatagtctgaaacgacatttcatacttgtacagacgcatgcattcttgaaatattgctctctctttctaaatagcgcgaatccgtcgctgtgcatttaggacatctcagtcgcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaaatgtacaagtatgaaatgtcgtttatgattatctttctagggttaa

SEQ ID NO: 54 - DNA, 30 bp, synthetic construct
  source 1..30: mol_type = other DNA
ccctagaaagatagtcaaacgacatttcat

SEQ ID NO: 55 - DNA, 30 bp, synthetic construct
  source 1..30: mol_type = other DNA
ccctagaaagataatcaaacgacatttcat

SEQ ID NO: 56 - DNA, 335 bp, synthetic construct
  source 1..335: mol_type = other DNA
ttaaccctagaaagatagtcaaacgacatttcatgacgcatgcattcttgaaatattgctctctctttctaaatagcgcgaatccgtcgctgtgcatttaggacatctcagtcgcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaaaatgaaatgtcgtttgattatctttctagggttaa

SEQ ID NO: 57 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 58 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 59 - protein, 1082 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSGGGGSSFVPNKEQTRTVLIFCFHLKKTAAESHRMLVEAFGEQVPTVKTCERWFQRFKSGDF

SEQ ID NO: 60 - protein, 1135 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSGGGGSSFVPNKEQTRTVLIFCFHLKKTAAESHRMLVEAFGEQVPTVKTCERWFQRFKSGDFDVDDKEHGKPPKRYEDAELQALLDEDDAQTQKQLAEQLEVSQQAVSNRLREMG

SEQ ID NO: 61 - protein, 1082 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSGGGGSSFVPNKEQTRTVLIFCFHLKKTAAESHRMLVEAFGEQVPTVKTCERWFQRFKSGDF

SEQ ID NO: 62 - protein, 1135 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSGGGGSSFVPNKEQTRTVLIFCFHLKKTAAESHRMLVEAFGEQVPTVKTCERWFQRFKSGDFDVDDKEHGKPPKRYEDAELQALLDEDDAQTQKQLAEQLEVSQQAVSNRLREMG

SEQ ID NO: 63 - DNA, 365 bp, synthetic construct
  source 1..365: mol_type = other DNA
ttaaccctagaaagatagtctgcgtaaaattgacgcatgcattcttgaaatattgctctctctttctaaatagcgcgaatccgtcgctgtgcatttaggacatctcagtcgcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaaacatgcgtcaattttacgcatgattatctttaacgtacgtcacaatatgattatctttctagggttaa

SEQ ID NO: 64 - DNA, 4401 bp, synthetic construct
  source 1..4401: mol_type = other DNA
ctagcggtctctttaaccctagaaagatagtctgcgtaaaattgacgcatgcattcttgaaatattgctctctctttctaaatgacgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcggcctccaaggcctgcaggagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtttagtcctgcaggtcggccgccacgaccggtgccaccatgactgaatacaaaccaactgttcgcctggcaactcgtgatgatgttccacgtgcagttcgcaccctggctgctgcatttgctgactaccctgcaacccgtcacactgtggacccagaccgccacattgaacgtgtgactgaactgcaggagctgttcctgacccgtgtgggcctggacattggcaaagtgtgggtggcagatgatggtgctgctgtggcagtgtggaccacccctgaatctgttgaagctggtgcagtgtttgctgagattggcccacgcatggcagaactgtctggcagccgcctggcagcacaacagcagatggaaggtctgctggcaccacaccgcccaaaagaacctgcttggttcctggcaactgtgggtgtgagccctgaccaccagggtaagggcctgggctctgcagtggtgctgcctggtgtggaagcagctgaacgtgcaggtgtgcctgctttcctggagacctcagctccacgcaacctgcctttctatgaacgcctgggcttcactgtgactgctgatgtggaagtgccagaaggcccacgcacttggtgcatgactcgcaaaccaggtgctagccttggctccggcgagggcaggggaagtcttctaacatgcggggacgtggaggaaaatcccggcccactcggctacccctacgacgtgcccgactacgccagcggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaactagatgctgtcgaccgatgcccttgagagccttcaacccagtcagctccttccggtgggcgcggggcaatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgactatcgtcgccgcacttattaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaagagacctgtacatacctaatgatttttatcaaaatcattaagttaaggtagatacacatcttgtcatatgatcaaatggtttcgccaaaaatcaataatcagacaacaagatgtgcgaactcgatattttacacgactctctttaccaattctaccacaacgtggatcagcagttcaacctgttgatagtacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccaaaaaaaagcccgctcattaggcgggctgcgcccttaagg

SEQ ID NO: 65 - DNA, 63 bp, synthetic construct
  source 1..63: mol_type = other DNA
ccctagaaagataatcatattgtgacgtacgttaaagataatcatgcgtaaaattgacgcatg

SEQ ID NO: 66 - protein, 326 aa, synthetic construct
LTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALE

SEQ ID NO: 67 - protein, 326 aa, synthetic construct
LTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALE

SEQ ID NO: 68 - protein, 555 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEP

SEQ ID NO: 69 - protein, 554 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEE

SEQ ID NO: 70 - protein, 553 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTE

SEQ ID NO: 71 - protein, 552 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDST

SEQ ID NO: 72 - protein, 551 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDS

SEQ ID NO: 73 - protein, 550 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDD

SEQ ID NO: 74 - protein, 549 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSD

SEQ ID NO: 75 - protein, 547 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGT

SEQ ID NO: 76 - protein, 546 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPG

SEQ ID NO: 77 - protein, 545 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVP

SEQ ID NO: 78 - protein, 544 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEV

SEQ ID NO: 79 - protein, 543 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKE

SEQ ID NO: 80 - protein, 542 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPK

SEQ ID NO: 81 - protein, 541 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILP

SEQ ID NO: 82 - protein, 540 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNIL

SEQ ID NO: 83 - protein, 539 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNI

SEQ ID NO: 84 - protein, 538 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISN

SEQ ID NO: 85 - protein, 501 aa, synthetic construct
NKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 86 - protein, 5 aa, synthetic construct
GGGGS

SEQ ID NO: 87 - protein, 511 aa, synthetic construct
TLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 88 - protein, 510 aa, synthetic construct
LPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 89 - protein, 509 aa, synthetic construct
PQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 90 - protein, 508 aa, synthetic construct
QRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 91 - protein, 507 aa, synthetic construct
RTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 92 - protein, 506 aa, synthetic construct
TIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 93 - protein, 505 aa, synthetic construct
IRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 94 - protein, 504 aa, synthetic construct
RGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 95 - protein, 503 aa, synthetic construct
GKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 96 - protein, 502 aa, synthetic construct
KNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 97 - protein, 501 aa, synthetic construct; identical to SEQ ID NO: 85

SEQ ID NO: 98 - protein, 500 aa, synthetic construct
KHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 99 - protein, 499 aa, synthetic construct
HCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 100 - protein, 498 aa, synthetic construct
CWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 101 - protein, 497 aa, synthetic construct
WSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 102 - protein, 496 aa, synthetic construct
STSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 103 - protein, 495 aa, synthetic construct
TSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 104 - protein, 494 aa, synthetic construct
SKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 105 - protein, 493 aa, synthetic construct
KSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 106 - protein, 492 aa, synthetic construct
STRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 107 - protein, 491 aa, synthetic construct
TRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 108 - protein, 511 aa, synthetic construct
TLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 109 - protein, 510 aa, synthetic construct
LPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 110 - protein, 509 aa, synthetic construct
PQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 111 - protein, 508 aa, synthetic construct
QRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 112 - protein, 507 aa, synthetic construct
RTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 113 - protein, 506 aa, synthetic construct
TIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 114 - protein, 505 aa, synthetic construct
IRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 115 - protein, 504 aa, synthetic construct
RGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 116 - protein, 503 aa, synthetic construct
GKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 117 - protein, 502 aa, synthetic construct
KNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 118 - protein, 501 aa, synthetic construct
NKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 119 - protein, 500 aa, synthetic construct
KHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 120 - protein, 499 aa, synthetic construct
HCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 121 - protein, 498 aa, synthetic construct
CWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 122 - protein, 497 aa, synthetic construct
WSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 123 - protein, 496 aa, synthetic construct
STSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 124 - protein, 495 aa, synthetic construct
TSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 125 - protein, 494 aa, synthetic construct
SKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 126 - protein, 493 aa, synthetic construct
KSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 127 - protein, 492 aa, synthetic construct
STRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 128 - protein, 491 aa, synthetic construct
TRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 129 - protein, 511 aa, synthetic construct
TLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 130 - protein, 510 aa, synthetic construct
LPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 131 - protein, 509 aa, synthetic construct
PQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 132 - protein, 508 aa, synthetic construct
QRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 133 - protein, 507 aa, synthetic construct
RTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 134 - protein, 506 aa, synthetic construct
TIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 135 - protein, 505 aa, synthetic construct
IRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 136 - protein, 504 aa, synthetic construct
RGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 137 - protein, 503 aa, synthetic construct
GKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 138 - protein, 502 aa, synthetic construct
KNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 139 - protein, 501 aa, synthetic construct
NKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 140 - protein, 500 aa, synthetic construct
KHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 141 - protein, 499 aa, synthetic construct
HCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 142 - protein, 498 aa, synthetic construct
CWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 143 - protein, 497 aa, synthetic construct
WSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 144 - protein, 496 aa, synthetic construct
STSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 145 - protein, 495 aa, synthetic construct
TSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 146 - protein, 494 aa, synthetic construct
SKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 147 - protein, 493 aa, synthetic construct
KSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 148 - protein, 492 aa, synthetic construct
STRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 149 - protein, 491 aa, synthetic construct
TRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 150 - protein, 511 aa, synthetic construct
TLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 151 - protein, 510 aa, synthetic construct
LPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 152 - protein, 509 aa, synthetic construct
PQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 153 - protein, 508 aa, synthetic construct
QRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 154 - protein, 507 aa, synthetic construct
RTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 155 - protein, 506 aa, synthetic construct
TIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 156 - protein, 505 aa, synthetic construct
IRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 157 - protein, 504 aa, synthetic construct
RGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 158 - protein, 503 aa, synthetic construct
GKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 159 - protein, 502 aa, synthetic construct
KNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 160 - protein, 501 aa, synthetic construct
NKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 161 - protein, 500 aa, synthetic construct
KHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 162 - protein, 499 aa, synthetic construct
HCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 163 - protein, 498 aa, synthetic construct
CWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 164 - protein, 497 aa, synthetic construct
WSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 165 - protein, 496 aa, synthetic construct
STSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 166 - protein, 495 aa, synthetic construct
TSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 167 - protein, 494 aa, synthetic construct
SKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 168 - protein, 493 aa, synthetic construct
KSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 169 - protein, 492 aa, synthetic construct
STRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 170 - protein, 491 aa, synthetic construct
TRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 171 - protein, 18 aa, synthetic construct
ILPNEVPGTSDDSTEEPV

SEQ ID NO: 172 - DNA, 16 bp, synthetic construct
  source 1..16: mol_type = other DNA
ccctagaaagatagtc

SEQ ID NO: 173 - DNA, 16 bp, synthetic construct
  source 1..16: mol_type = other DNA
ccctagaaagatagtc

SEQ ID NO: 174 - protein, 18 aa, synthetic construct
ILPKEVPGTSDDSTEEPV

(19) United States
(12) Patent Application Publication
(10) Pub. No.: US 2026/0085332 A1
(43) Pub. Date: Mar. 26, 2026

(54) CHIMERIC TRANSPOSASES AND USES THEREOF

(71) Applicant: Poseida Therapeutics, Inc., San Diego, CA (US)

(72) Inventors: Joseph S. LUCAS, San Diego, CA (US); Blair B. MADISON, San Diego, CA (US); Olga BATALOV, San Diego, CA (US)

(21) Appl. No.: 19/349,664

(22) Filed: Oct. 3, 2025

Related U.S. Application Data
(63) Continuation of application No. PCT/US2024/022978, filed on Apr. 4, 2024.
(60) Provisional application No. 63/494,297, filed on Apr. 5, 2023.

(51) Int. Cl. C12N 15/90; C12N 9/22

(52) U.S. Cl. CPC C12N 15/907; C12N 9/22; C12N 2800/90

(57) ABSTRACT

Provided herein are chimeric transposases and chimeric site-specific fusion proteins, polynucleotides encoding the chimeric transposases and chimeric site-specific fusion proteins, and vectors and transposons comprising the polynucleotides. Also provided are methods of making the chimeric transposases and chimeric fusion proteins, cells that are modified using the chimeric transposases or chimeric site-specific fusion proteins provided herein and methods using such cells.

### CROSS-REFERENCE TO RELATED APPLICATIONS

The present application is a continuation of International Patent Application No. PCT/US2024/022978, filed Apr. 4, 2024, which claims the benefit of U.S. Provisional Patent Application No. 63/494,297, filed Apr. 5, 2023, each of which is incorporated herein by reference in their entireties.

### REFERENCE TO SEQUENCE LISTING SUBMITTED ELECTRONICALLY

The instant application contains a Sequence Listing, which has been submitted electronically in XML file format, and is herein incorporated by reference into the specification in its entirety. The XML file containing the Sequence Listing XML is named “000218-0140-101-SL.xml,” was created on Oct. 3, 2025 and is 234,180 bytes in size.

### FIELD

This disclosure generally relates to chimeric transposases, in particular, chimeric transposases, chimeric site-specific transposase fusion proteins comprising the chimeric transposases and DNA targeting domains, and chimeric transposon inverted repeat (ITR) polynucleotides. Also provided are methods of use of the chimeric site-specific transposase fusion proteins for site-specific transposition.

### BACKGROUND

Transposases may be used to introduce non-endogenous DNA sequences into genomic DNA, and are in many ways advantageous to other methods gene editing. However, there remains an unmet need for site-specific transposases for use in e.g., gene editing.

In their natural form, the PiggyBac transposon and related family members harbor a single open reading frame encoding their respective transposase. Inverted Terminal Repeat (ITR) sequences are present at the two ends of the transposons and act as binding sites for the transposases. Immediately outside of the ITRs, a four-nucleotide sequence, TTAA, acts as the cleavage site, allowing the transposons to jump from one location in a genome into another TTAA site through a cut-and-paste mechanism.

When used for genome editing purposes, the transposase is often removed from the transposon and expressed from a separate plasmid or mRNA. This leaves an empty transposon where a “cargo” or “payload” of interest, flanked by the ITRs and TTAA sequences, can be encoded. This synthetic transposon can be delivered to cells along with the transposase, where the transposase catalyzes the transposition of the transposon into a TTAA site in the genome of the cell.

PiggyBac transposase and related family members contain two distinct DNA binding domains that interact with the Inverted Terminal (ITR) sequences of the transposon. The DNA binding and dimerization domain (DDBD) binds sequence proximal to the TTAA sites flanking the transposon while the Cysteine Rich Domain (CRD) at the C-terminus of the protein binds Repeat (ITR) sequence distal to the TTAA sites. Upon dimerization of the transposase, the DDBDs bind symmetrically to the ITRs, with the DDBD of one monomer binding the left end (LE) ITR and the DDBD of the second monomer binding the right end (RE) ITR. Interestingly, the two CRDs of the dimer bind asymmetrically with both binding to the LE ITR. Based on ITR sequence identity, it has been proposed that a second dimer of piggyBac transposase may bind distal to the first dimer. Both CRDs of this second dimer have predicted binding sites within the RE ITR. While the proximal dimer catalyzes the transposition reaction, the role of the distal dimer remains unclear.

The asymmetrical binding of the two CRD sequences of piggyBac transposase is not a feature of all transposase families. For instance, MosI, a member of the mariner/Tcl family of transposases interacts with its ITRs as a symmetrical dimer, as observed in its crystal structure (see, e.g., Morris et al., Elife 2016 May 25; 5:e15537. doi: 10.7554/eLife. 15537). A transposase that functions as a symmetrical dimer could have several beneficial features over a transposase that functions as an asymmetrical pair of dimers (e.g., piggyBac transposase), including shorter ITR sequences and fewer transposase molecules needed to complete transposition.

### SUMMARY

In one aspect, provided herein is a chimeric transposase comprising, in N-terminal to C-terminal order: (i) a target-specific DNA binding domain, (ii) a truncated Super piggyBac (SPB) transposase comprising a C-terminal Cysteine Rich Domain (CRD) deletion within amino acid residues 535-594 of the sequence of the SPB comprising the sequence set forth in SEQ ID NO: 1, and (iii) one or more MosI DNA binding domain(s). In some embodiments, the truncated PB transposase of (i) comprises the sequence set forth in any one of SEQ ID NOs.: 68-84. In some embodiments, the truncated PB transposase comprises one or more hyperactive mutation. In some embodiments, the one or more hyperactive mutation is an amino acid substitution selected from I30V, S103P, G165S, M226F, M282V, S509G, N538K or N571S of SEQ ID No: 1.

In some embodiments, the PB transposase comprising the CRD deletion further comprises an in-frame N-terminal nuclear localization sequence (NLS) comprising the amino acid sequence of SEQ ID NO: 39. In some embodiments, the chimeric transposase comprises two MosI DNA binding domains. In some embodiments, the two MosI DNA binding domains comprise the amino acid sequence set forth in SEQ ID NO: 6. In some embodiments, the chimeric transposase comprises one MosI DNA binding domain. In some embodiments, the one MosI DNA binding domain comprises the amino acid sequence set forth in SEQ ID NO: 8.

In some embodiments, the PB transposase and the one or more MosI DNA binding domain(s) are connected by a linker sequence. In some embodiments, the linker sequence is GGGGS (SEQ ID NO: 86). In some embodiments, the target-specific DNA binding domain is a zing finger domain or a TAL array.

In another aspect, provided herein is a polynucleotide comprising a nucleic acid sequence encoding a chimeric transposase described herein.

In another aspect, provided herein is a vector comprising a polynucleotide provided herein.

In another aspect, provided herein is a chimeric transposon inverted terminal repeat (ITR) polynucleotide, comprising in the 5′ to 3′ direction: (i) a polynucleotide comprising nucleotides 1-16 of the nucleic acid sequence set forth in SEQ ID NO: 12 and (ii) a polynucleotide comprising two MosI DNA binding sites. In some embodiments, the polynucleotide comprising two MosI DNA binding sites is fused in the reverse orientation. In some embodiments, the polynucleotide comprising two MosI DNA binding sites comprises the nucleic acid sequence of SEQ ID NOs: 13 or 14. In some embodiments, the chimeric transposon ITR polynucleotide comprises the nucleic acid sequence of SEQ ID NOs: 15 or 16.

In some embodiments, the chimeric transposon ITR polynucleotide further comprises one or two additional nucleotides between the polynucleotide comprising nucleotides 1-16 of the nucleic acid sequence set forth in SEQ ID NO: 12 and the polynucleotide comprising two MosI binding sites. In some embodiments, the chimeric transposon ITR polynucleotide comprises the nucleic acid sequence of SEQ ID NO: 17. In some embodiments, the chimeric transposon ITR polynucleotide comprises the nucleic acid sequence of SEQ ID NO: 18.

In another aspect, provided herein is a vector comprising a chimeric transposon ITR polynucleotide provided herein.

In another aspect, provided herein is a transposon comprising a chimeric transposon ITR polynucleotide described herein.

In another aspect, provided herein is a chimeric transposon ITR polynucleotide, comprising in the 5′ to 3′ direction: (i) a polynucleotide comprising nucleotides 1-16 of the nucleic acid sequence set forth in SEQ ID NO: 65 and (ii) a polynucleotide comprising two MosI DNA binding sites. In some embodiments, the polynucleotide comprising two MosI DNA binding sites is fused in the reverse orientation. In some embodiments, the polynucleotide comprising two MosI DNA binding sites comprises the nucleic acid sequence of SEQ ID NOs: 13 or 14. In some embodiments, the chimeric transposon ITR polynucleotide comprises the nucleic acid sequence of SEQ ID NOs: 19 or 20. In some embodiments, the chimeric transposon ITR polynucleotide further comprises one or two additional nucleotides between the polynucleotide comprising nucleotides 1-16 of the nucleic acid sequence set forth in SEQ ID NO: 65 and the polynucleotide comprising two MosI DNA binding sites. In some embodiments, the chimeric transposon ITR polynucleotide comprises the nucleic acid sequence of SEQ ID NOs: 21 or 22.

In another aspect, provided herein is a vector comprising a chimeric transposon ITR polynucleotide described herein.

In another aspect, provided herein is a transposon comprising a chimeric transposon ITR polynucleotides described herein.

In another aspect, provided herein is a chimeric site-specific transposase fusion proteins comprising, in N-terminal to C-terminal order: (i) a target-specific DNA binding domain, (ii) a piggyBac transposase comprising an N-terminal deletion relative to SEQ ID NO: 1, one or more integration-deficient mutations relative to SEQ ID NO: 1 and a C-terminal Cysteine Rich Domain (CRD) deletion within residues 535-594 of the amino acid sequence SEQ ID NO: 1; and (iii) one or more MosI DNA binding domain(s). In some embodiments, the target-specific DNA binding domain is a TAL or a zinc finger motif (ZFM).

In some embodiments, the truncated PB transposase comprises the sequence set forth in any one SEQ ID NOs: 68-84. In some embodiments, the N-terminal deletion of the truncated PB transposase is a deletion of amino acid residues 1-85, 1-86, 1-87, 1-88, 1-89, 1-90, 1-91, 1-92, 1-93, 1-94, 1-95, 1-96, 1-97, 1-98, 1-99, 1-100, 1-101, 1-102, or 1-103 of SEQ ID NO: 1. In some embodiments, the N-terminal deletion of the truncated PB transposase is a deletion of amino acid residues 1-93. In some embodiments, the PB transposase comprises one or more hyperactive mutation(s). In some embodiments, the one or more hyperactive mutation is an amino acid substitution selected from G165S, M226F, M282V and N538K of SEQ ID NO: 1 with numbering beginning at residue 5. In some embodiments, the one or more integration deficient mutation of the truncated PB transposase is an amino acid substitution selected from R372A, K375A, and D450N of SEQ ID NO: 1. In some embodiments, the N-terminal deleted truncated PB transposase comprises the amino acid sequence of SEQ ID NO: 85.

In some embodiments, the PB transposase comprising a C-terminal Cysteine Rich Domain (CRD) deletion further comprises an in-frame N-terminal nuclear localization sequence (NLS), wherein the NLS comprises the amino acid sequence set forth in SEQ ID NO: 39. In some embodiments, the chimeric site-specific transposase fusion protein comprises two MosI DNA binding domains. In some embodiments, the two MosI DNA binding domains comprises the amino acid sequence set forth in SEQ ID NO: 6. In some embodiments, the chimeric site-specific transposase fusion protein comprises one MosI DNA binding domain. In some embodiments, the one MosI DNA binding domain comprises the amino acid sequence set forth in SEQ ID NO: 8.

In some embodiments, the PB transposase and the one or more MosI DNA binding domain(s) are connected by a linker sequence. In some embodiments, the linker sequence is GGGGS (SEQ ID NO: 86).

In some embodiments, the target-specific DNA binding domain is a zinc finger domain or a TAL array.

In another aspect, provided herein is a LINE1-targeting chimeric site-specific transposase fusion protein comprising, in the N-terminal to C-terminal direction, (i) a left or a right LINE1 TAL-targeting DNA binding domain, (ii) a linker sequence, and (iii) a chimeric PB:MosI transposase. In some embodiments, the chimeric PB:MosI transposase comprises the amino acid sequence set forth in SEQ ID NOs: 9 or 7. In some embodiments, the left or the right LINE1 TAL-targeting DNA binding domain comprises the amino acid sequence set forth in SEQ ID NOs: 66 or 67.

In another aspect, provided herein is a polynucleotide comprising a nucleic acid sequence encoding a chimeric site-specific transposase fusion protein described herein.

In another aspect, provided herein is a vector comprising a nucleic acid sequence encoding a chimeric site-specific transposase fusion protein described herein.

In another aspect, provided herein is a transposon, comprising (i) a chimeric LE PB:MosI ITR polynucleotide and (ii) a chimeric RE PB:MosI ITR polynucleotide, wherein the LE PB:MosI ITR polynucleotide and an RE PB:MosI ITR polynucleotide comprise the same nucleic acid sequence. In some embodiments, the chimeric LE PB:MosI ITR polynucleotide and the chimeric RE PB:MosI ITR polynucleotide each comprise the nucleic acid sequence of any one of SEQ ID NOs: 15-22.

In another aspect, provided herein is a transposon, comprising (i) a chimeric LE PB:MosI ITR polynucleotide comprising the nucleic acid sequence of SEQ ID NO: 54 and (ii) a chimeric RE PB:MosI ITR polynucleotide comprising the nucleic acid sequence of SEQ ID NO: 55.

In another aspect, provided herein is a transposon comprising the nucleic acid sequence of SEQ ID NO: 56.

In another aspect, provided herein is a method of integrating a transgene into a genomic target site of a cell, the method comprising introducing into the cell a chimeric transposase disclosed herein and a transposon, wherein the transposon comprises, in 5′ to 3′ order: a 5′ITR, the transgene, and a 3′ ITR, wherein the 5′ITR is a chimeric transposon inverted terminal repeat (ITR) described herein and the 3′UTR is a chimeric transposon inverted terminal repeat (ITR) described herein. In some embodiments, the 5′ITR chimeric transposon inverted terminal repeat comprises the nucleic acid sequence set forth in SEQ ID NO: 15. In some embodiments, the 3′ITR chimeric transposon inverted terminal repeat comprises the nucleic acid sequence set forth in SEQ ID NO: 21. In some embodiments, the transposon further comprises an exogenous promoter between the 5′ ITR and the transgene.

In some embodiments, the transgene encodes a detectable marker. In some embodiments, the detectable marker is GFP.

In some embodiments, the genomic target site is located in a repetitive element. In some embodiments, the repetitive element is a LINE element.

In another aspect, provided herein is a method for site-specific transposition of a DNA molecule into the genome of a cell, comprising introducing into the cell: (a) a nucleic acid encoding a chimeric site-specific transposase fusion protein comprising a DNA binding domain and a chimeric transposase comprising, in N-terminal to C-terminal order: a piggyBac transposase comprising a C-terminal Cysteine Rich Domain (CRD) deletion and one or more MosI DNA binding domain(s); wherein the fusion protein is expressed in the cell; and a (b) DNA molecule comprising a transposon comprising a chimeric LE transposon ITR polynucleotide and a chimeric RE transposon ITR polynucleotide; wherein the expressed chimeric site-specific transposase fusion protein integrates the transposon by site-specific transposition into the TTAA sequence of cellular genome.

In another aspect, provided herein is a method for generating an engineered cell by site-specific transposition, comprising introducing into the cell: (a) a nucleic acid encoding a chimeric site-specific transposase fusion protein comprising a DNA binding domain and a chimeric transposase comprising, in N-terminal to C-terminal order: a piggyBac transposase comprising a C-terminal Cysteine Rich Domain (CRD) deletion and one or more MosI DNA binding domain(s); wherein the chimeric site-specific transposase fusion protein is expressed in the cell; and (b) a DNA molecule comprising a transposon comprising a chimeric LE transposon ITR polynucleotide and a chimeric RE transposon ITR polynucleotide; wherein the expressed chimeric site-specific transposase fusion protein integrates the transposon by site-specific transposition into the TTAA sequence into the genome of the cell thereby generating the engineered cell.

### DETAILED DESCRIPTION

Provided herein are chimeric transposases and chimeric site-specific fusion proteins, in particular chimeric piggyBac: MosI (PB:MosI) transposases and chimeric PB:MosI site-specific fusion proteins, polynucleotides encoding the chimeric transposases and chimeric site-specific fusion proteins, and vectors and transposons comprising the polynucleotides. Also provided are methods of making the chimeric transposases and chimeric fusion proteins, cells that are modified using the chimeric transposases or chimeric site-specific fusion proteins provided herein and methods using such cells.

Also provided herein are chimeric transposon inverted terminal repeat (ITR) sequences. In some embodiments, the chimeric transposon inverted terminal repeat (ITR) sequences are chimeric PB:MosI transposon left end inverted terminal repeat (ITR) sequences. In some embodiments, the chimeric transposon inverted terminal repeat (ITR) sequences are chimeric PB:MosI transposon right end inverted terminal repeat (ITR) sequences. Also provided are transposons comprising the chimeric left end and right end ITRs and methods of using the transposons comprising the chimeric transposon left end and right end ITR sequences provided herein in methods to transpose cells and use thereof.

Chimeric piggyBac: MosI Transposases

In one aspect, provided herein are chimeric transposases comprising, in N-terminal to C-terminal order: a piggyBac transposase comprising a C-terminal Cysteine Rich Domain (CRD) deletion and one or more MosI DNA binding domain(s).

An exemplary sequence of a wildtype PB transposase is set forth in SEQ ID NO: 1.

(SEQ ID NO: 1)
MGSSLDDEHILSALLQSDDELVGEDSDSEISDHVSEDDVQSDTEEAFIDE
VHEVQPTSSGSEILDEQNVIEQPGSSLASNRILTLPQRTIRGKNKHCWST
SKSTRRSRVSALNIVRSQRGPTRMCRNIYDPLLCFKLFFTDEIISEIVKW
TNAEISLKRRESMTGATFRDTNEDEIYAFFGILVMTAVRKDNHMSTDDLF
DRSLSMVYVSVMSRDRFDFLIRCLRMDDKSIRPTLRENDVFTPVRKIWDL
FIHQCIQNYTPGAHLTIDEQLLGFRGRCPFRMYIPNKPSKYGIKILMMCD
SGTKYMINGMPYLGRGTQTNGVPLGEYYVKELSKPVHGSCRNITCDNWFT
SIPLAKNLLQEPYKLTIVGTVRSNKREIPEVLKNSRSRPVGTSMFCFDGP
LTLVSYKPKPAKMVYLLSSCDEDASINESTGKPQMVMYYNQTKGGVDTLD
QMCSVMTCSRKTNRWPMALLYGMINIACINSFIIYSHNVSSKGEKVQSRK
KFMRNLYMSLTSSFMRKRLEAPTLKRYLRDNISNILPNEVPGTSDDSTEE
PVMKKRTYCTYCPSKIRRKANASCKKCKKVICREHNIDMCQSCF

In one aspect, provided herein are chimeric transposases comprising, in N-terminal to C-terminal order: (i) a piggyBac transposase comprising a C-terminal Cysteine Rich Domain (CRD) deletion and (ii) one or more MosI DNA binding domain(s). In some embodiments, the PB CRD consists of residues 553-594 of the 594 amino acid PB transposase protein (i.e., in some embodiments, the PB CRD comprises the amino acid sequence set forth in SEQ ID NO: 2). The CRD domain may be attached to the rest of the N-terminus of the transposase through a linker sequence. In some embodiments, the linker sequence spans residues 535-552 of the PB transposase sequence (inclusive of residues 535 and 552). In some embodiments, the linker comprises the amino acid sequence ILPNEVPGTSDDSTEEPV (SEQ ID NO: 171). In some embodiments, the linker comprises the amino acid sequence ILPKEVPGTSDDSTEEPV (SEQ ID NO: 174).

In some embodiments, the C-terminal CRD deletion of the piggyBac transposase is a deletion of C-terminal amino acid residues 545-594 of the PB transposase amino acid sequence set forth in SEQ ID NO: 1. In such embodiments, the resulting truncated piggyBac transposase comprises the amino acid sequence:

(SEQ ID NO: 3)
MGSSLDDEHILSALLQSDDELVGEDSDSEISDHVSEDDVQSDTEEAFIDE
VHEVQPTSSGSEILDEQNVIEQPGSSLASNRILTLPQRTIRGKNKHCWST
SKSTRRSRVSALNIVRSQRGPTRMCRNIYDPLLCFKLFFTDEIISEIVKW
TNAEISLKRRESMTGATFRDTNEDEIYAFFGILVMTAVRKDNHMSTDDLF
DRSLSMVYVSVMSRDRFDFLIRCLRMDDKSIRPTLRENDVFTPVRKIWDL
FIHQCIQNYTPGAHLTIDEQLLGFRGRCPFRMYIPNKPSKYGIKILMMCD
SGTKYMINGMPYLGRGTQTNGVPLGEYYVKELSKPVHGSCRNITCDNWFT
SIPLAKNLLQEPYKLTIVGTVRSNKREIPEVLKNSRSRPVGTSMFCFDGP
LTLVSYKPKPAKMVYLLSSCDEDASINESTGKPQMVMYYNQTKGGVDTLD
QMCSVMTCSRKTNRWPMALLYGMINIACINSFIIYSHNVSSKGEKVQSRK
KFMRNLYMSLTSSFMRKRLEAPTLKRYLRDNISNILPNEVPGTS

In some embodiments, a PB transpose comprising a CRD deletion described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 3. In some embodiments, a PB transpose comprising a CRD deletion described herein comprises the amino acid sequence set forth in SEQ ID NO: 3 with one, two, three, four of five conservative amino acid substitutions. In some embodiments, a PB transpose comprising a CRD deletion described herein comprises the amino acid sequence set forth in SEQ ID NO: 3.

MosI DNA binding domain(s) used to generate chimeric PB:MosI transposases comprising this C-terminal CRD deletion may be joined to amino acid S544 of the PB transposase sequence set forth in SEQ ID NO: 1. The resulting chimeric transposase is referred to herein as a “chimeric S544 PB:MosI transposase.” In some embodiments, the S544 PB transposase sequence and the MosI DNA binding domain(s) are connected by a linker sequence. In some embodiments, the linker sequence is GGGGS (SEQ ID NO: 86).

In some embodiments, the C-terminal Cysteine Rich Domain (CRD) deletion of the PB transposase is a deletion of amino acid residues 553-594 of the PB transposase amino acid sequence set forth in SEQ ID NO: 1. In such embodiments, the resulting truncated piggyBac transposase comprises the amino acid sequence:

(SEQ ID NO: 4)
MGSSLDDEHILSALLQSDDELVGEDSDSEISDHVSEDDVQSDTEEAFIDE
VHEVQPTSSGSEILDEQNVIEQPGSSLASNRILTLPQRTIRGKNKHCWST
SKSTRRSRVSALNIVRSQRGPTRMCRNIYDPLLCFKLFFTDEIISEIVKW
TNAEISLKRRESMTGATFRDTNEDEIYAFFGILVMTAVRKDNHMSTDDLF
DRSLSMVYVSVMSRDRFDFLIRCLRMDDKSIRPTLRENDVFTPVRKIWDL
FIHQCIQNYTPGAHLTIDEQLLGFRGRCPFRMYIPNKPSKYGIKILMMCD
SGTKYMINGMPYLGRGTQTNGVPLGEYYVKELSKPVHGSCRNITCDNWFT
SIPLAKNLLQEPYKLTIVGTVRSNKREIPEVLKNSRSRPVGTSMFCFDGP
LTLVSYKPKPAKMVYLLSSCDEDASINESTGKPQMVMYYNQTKGGVDTLD
QMCSVMTCSRKTNRWPMALLYGMINIACINSFIIYSHNVSSKGEKVQSRK
KFMRNLYMSLTSSFMRKRLEAPTLKRYLRDNISNILPNEVPGTSDDSTEE
PV

In some embodiments, a PB transpose comprising a CRD deletion described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 4. In some embodiments, a PB transpose comprising a CRD deletion described herein comprises the amino acid sequence set forth in SEQ ID NO: 4 with one, two, three, four of five conservative amino acid substitutions. In some embodiments, a PB transpose comprising a CRD deletion described herein comprises the amino acid sequence set forth in SEQ ID NO: 4.

Alternatively, MosI DNA binding domain(s) used to generate chimeric PB:MosI transposases comprising this C-terminal CRD deletion may be joined to amino acid V552 of the piggyBac transposase sequence set forth in SEQ ID NO: 1. The resulting chimeric transposase is referred to herein as a “chimeric V552 PB:MosI transposase.” In some embodiments, the V552 PB transposase sequence and the MosI DNA binding domain(s) are connected by a linker sequence. In some embodiments, the linker sequence is GGGGS (SEQ ID NO: 86).

In some embodiments, the truncated PB transposase comprises one or more hyperactive mutation. Example of hyperactive mutations include I30V, S103P, G165S, M226F, M282V, S509G, N538K and N571S of SEQ ID NO.: 1. In some embodiments, the truncated piggyBac transposase comprises at least 4 hyperactive mutations selected from I30V, G165S, M226F, M282V and N538K of SEQ ID NO: 1.

In some embodiments, a truncated PB transposase described herein comprises the sequence of SEQ ID NO: 1 and one of the following mutations: I30V, S103P, G165S, M226F, M282V, S509G, N538K and N571S. In some embodiments, a truncated PB transposase described herein comprises the sequence of SEQ ID NO: 1 and two of the following mutations: I30V, S103P, G165S, M226F, M282V, S509G, N538K and N571S. In some embodiments, a truncated PB transposase described herein comprises the sequence of SEQ ID NO: 1 and three of the following mutations: I30V, S103P, G165S, M226F, M282V, S509G, N538K and N571S. In some embodiments, a truncated PB transposase described herein comprises the sequence of SEQ ID NO: 1 and four of the following mutations: I30V, S103P, G165S, M226F, M282V, S509G, N538K and N571S. In some embodiments, a truncated PB transposase described herein comprises the sequence of SEQ ID NO: 1 and five of the following mutations: I30V, S103P, G165S, M226F, M282V, S509G, N538K and N571S. In some embodiments, a truncated PB transposase described herein comprises the sequence of SEQ ID NO: 1 and six of the following mutations: I30V, S103P, G165S, M226F, M282V, S509G, N538K and N571S. In some embodiments, a truncated PB transposase described herein comprises the sequence of SEQ ID NO: 1 and seven of the following mutations: I30V, S103P, G165S, M226F, M282V, S509G, N538K and N571S. In some embodiments, a truncated PB transposase described herein comprises the sequence of SEQ ID NO: 1 and the following mutations: I30V, S103P, G165S, M226F, M282V, S509G, N538K and N571S.

In some embodiments, a truncated PB transposase described herein comprises the sequence of SEQ ID NO: 3 and one of the following mutations: I30V, S103P, G165S, M226F, M282V, S509G, N538K and N571S. In some embodiments, a truncated PB transposase described herein comprises the sequence of SEQ ID NO: 3 and two of the following mutations: I30V, S103P, G165S, M226F, M282V, S509G, N538K and N571S. In some embodiments, a truncated PB transposase described herein comprises the sequence of SEQ ID NO: 3 and three of the following mutations: I30V, S103P, G165S, M226F, M282V, S509G, N538K and N571S. In some embodiments, a truncated PB transposase described herein comprises the sequence of SEQ ID NO: 3 and four of the following mutations: I30V, S103P, G165S, M226F, M282V, S509G, N538K and N571S. In some embodiments, a truncated PB transposase described herein comprises the sequence of SEQ ID NO: 3 and five of the following mutations: I30V, S103P, G165S, M226F, M282V, S509G, N538K and N571S. In some embodiments, a truncated PB transposase described herein comprises the sequence of SEQ ID NO: 3 and six of the following mutations: I30V, S103P, G165S, M226F, M282V, S509G, N538K and N571S. In some embodiments, a truncated PB transposase described herein comprises the sequence of SEQ ID NO: 3 and seven of the following mutations: I30V, S103P, G165S, M226F, M282V, S509G, N538K and N571S. In some embodiments, a truncated PB transposase described herein comprises the sequence of SEQ ID NO: 3 and the following mutations: I30V, S103P, G165S, M226F, M282V, S509G, N538K and N571S.

In some embodiments, a truncated PB transposase described herein comprises the sequence of SEQ ID NO: 4 and one of the following mutations: I30V, S103P, G165S, M226F, M282V, S509G, N538K and N571S. In some embodiments, a truncated PB transposase described herein comprises the sequence of SEQ ID NO: 4 and two of the following mutations: I30V, S103P, G165S, M226F, M282V, S509G, N538K and N571S. In some embodiments, a truncated PB transposase described herein comprises the sequence of SEQ ID NO: 4 and three of the following mutations: I30V, S103P, G165S, M226F, M282V, S509G, N538K and N571S. In some embodiments, a truncated PB transposase described herein comprises the sequence of SEQ ID NO: 4 and four of the following mutations: I30V, S103P, G165S, M226F, M282V, S509G, N538K and N571S. In some embodiments, a truncated PB transposase described herein comprises the sequence of SEQ ID NO: 4 and five of the following mutations: I30V, S103P, G165S, M226F, M282V, S509G, N538K and N571S. In some embodiments, a truncated PB transposase described herein comprises the sequence of SEQ ID NO: 4 and six of the following mutations: I30V, S103P, G165S, M226F, M282V, S509G, N538K and N571S. In some embodiments, a truncated PB transposase described herein comprises the sequence of SEQ ID NO: 4 and seven of the following mutations: I30V, S103P, G165S, M226F, M282V, S509G, N538K and N571S. In some embodiments, a truncated PB transposase described herein comprises the sequence of SEQ ID NO: 4 and the following mutations: I30V, S103P, G165S, M226F, M282V, S509G, N538K and N571S.

In some embodiments, the truncated piggyBac transposase domain is a Super piggyBac® transposase (SPB). Non-limiting examples of SPB transposases are described in detail in U.S. Pat. Nos. 6,218,182; 6,962,810; 8,399,643 and PCT Publication No. WO 2010/099296, each of which is incorporated herein by reference in its entirety, for examples of SPB transposases that may be used in the fusion proteins described herein.

Illustrative Super piggyBac transposase-MosI chimeric transposases are further described in Example 6.

In some embodiments, the truncated PB transposase sequence further comprises an in-frame N-terminus nuclear localization sequence (NLS). An illustrative wildtype SPB sequence comprising a NLS is shown in SEQ ID NO: 5 with the NLS shown in italics, hyperactive mutations shown in bold, and the Cysteine Rich Domain (CRD) deleted in the truncated PB transposase sequence underlined. The numbering of sequence of the SPB transposase domain for the purpose of describing deletions and mutations begins at residue 12 of SEQ ID NO: 5.

(SEQ ID NO: 5)
MAPKKKRKVGGGGSSLDDEHILSALLQSDDELVGEDSDSEVSDHVSEDDV
QSDTEEAFIDEVHEVQPTSSGSEILDEQNVIEQPGSSLASNRILTLPQRT
IRGKNKHCWSTSKSTRRSRVSALNIVRSQRGPTRMCRNIYDPLLCFKLFF
TDEIISEIVKWTNAEISLKRRESMTSATFRDTNEDEIYAFFGILVMTAVR
KDNHMSTDDLFDRSLSMVYVSVMSRDRFDFLIRCLRMDDKSIRPTLREND
VFTPVRKIWDLFIHQCIQNYTPGAHLTIDEQLLGFRGRCPFRVYIPNKPS
KYGIKILMMCDSGTKYMINGMPYLGRGTQTNGVPLGEYYVKELSKPVHGS
CRNITCDNWFTSIPLAKNLLQEPYKLTIVGTVRSNKREIPEVLKNSRSRP
VGTSMFCFDGPLTLVSYKPKPAKMVYLLSSCDEDASINESTGKPQMVMYY
NQTKGGVDTLDQMCSVMTCSRKTNRWPMALLYGMINIACINSFIIYSHNV
SSKGEKVQSRKKFMRNLYMSLTSSFMRKRLEAPTLKRYLRDNISNILPKE
VPGTSDDSTEEPVMKKRTYCTYCPSKIRRKANASCKKCKKVICREHNIDM
CQSCE.

The transposases described herein can be isolated or derived from an insect, a vertebrate, a crustacean or an urochordate as described in more detail in PCT Publication No. WO 2019/173636 and PCT/US2019/049816. In preferred aspects, the SPB transposase is isolated or derived from the insect Trichoplusia ni (GenBank Accession No. AAA87375), Macdunnoughia crassisigna (ABZ85926.1), or Bombyx mori (GenBank Accession No. BAD11135).

In some embodiments, the chimeric transposase comprises two MosI DNA binding domains. In some embodiments, each of the two MosI DNA binding domains comprises amino acid residues 3-111 of the MosI transposase (i.e., comprises the amino acid sequence set forth in SEQ ID NO: 6): SFVPNKEQTRTVLIFCFHLKKTAAESHRMLVEAFGEQVPTVKTCERWFQRFKSGDFDVD DKEHGKPPKRYEDAELQALLDEDDAQTQKQLAEQLEVSQQAVSNRLREMG (SEQ ID NO: 6). In some embodiments, each of the two MosI DNA binding domains comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 6. In some embodiments, each of the two MosI DNA binding domains comprises the amino acid sequence set forth in SEQ ID NO: 3 with one, two, three, four of five conservative amino acid substitutions.

In some embodiments, the chimeric transposase comprising a truncated piggyBac transposase domain comprising the amino acid sequence set forth in SEQ ID NO: 3 connected to the two MosI DNA binding domains, wherein each of the two MosI DNA binding domains comprises the sequence set forth in SEQ ID NO: 6. The two MosI DNA binding domains may be attached to the truncated PB transposase domain via a linker sequence, resulting in chimeric PB:MosI S544-111 transposase comprising the amino acid sequence set forth in SEQ ID NO: 7, with residues 3-111 of the MosI transposase highlighted in bold font, the PB domain shown underlined, and the linker sequence highlighted in italics:

(SEQ ID NO: 7)
MAPKKKRKVGGGGSSLDDEHILSALLQSDDELVGEDSDSEVSDHVSEDDV
QSDTEEAFIDEVHEVQPTSSGSEILDEQNVIEQPGSSLASNRILTLPQRT
IRGKNKHCWSTSKSTRRSRVSALNIVRSQRGPTRMCRNIYDPLLCFKLFF
TDEIISEIVKWTNAEISLKRRESMTSATFRDTNEDEIYAFFGILVMTAVR
KDNHMSTDDLFDRSLSMVYVSVMSRDRFDFLIRCLRMDDKSIRPTLREND
VFTPVRKIWDLFIHQCIQNYTPGAHLTIDEQLLGFRGRCPFRVYIPNKPS
KYGIKILMMCDSGTKYMINGMPYLGRGTQTNGVPLGEYYVKELSKPVHGS
CRNITCDNWFTSIPLAKNLLQEPYKLTIVGTVRSNKREIPEVLKNSRSRP
VGTSMFCFDGPLTLVSYKPKPAKMVYLLSSCDEDASINESTGKPQMVMYY
NQTKGGVDTLDQMCSVMTCSRKTNRWPMALLYGMINIACINSFIIYSHNV
SSKGEKVQSRKKFMRNLYMSLTSSFMRKRLEAPTLKRYLRDNISNILPKE
VPGTSGGGGSSFVPNKEQTRTVLIFCFHLKKTAAESHRMLVEAFGEQVPT
VKTCERWFQRFKSGDFDVDDKEHGKPPKRYEDAELQALLDEDDAQTQKQL
AEQLEVSQQAVSNRLREMG.

In some embodiments, a chimeric PB:MosI transpose comprising a CRD deletion described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 7. In some embodiments, a chimeric PB:MosI transpose comprising a CRD deletion described herein comprises the amino acid sequence set forth in SEQ ID NO: 7 with one, two, three, four of five conservative amino acid substitutions. In some embodiments a chimeric PB:MosI transpose comprising a CRD deletion described herein comprises the amino acid sequence set forth in SEQ ID NO: 7.

In some embodiments, the chimeric transposase comprises one MosI DNA binding domain. In some embodiments, the one MosI DNA binding domain comprises amino acid residues 3-58 of the MosI transposase, i.e., comprises the amino acid sequence set forth in SEQ ID NO: 8:

(SEQ ID NO: 8)
SFVPNKEQTRTVLIFCFHLKKTAAESHRMLVEAFGEQVPTVKTCERWFQR
FKSGDF.

In some embodiments, the chimeric transposase comprising a truncated piggyBac transposase domain comprising the amino acid sequence set forth in SEQ ID NO: 3 is connected to one MosI DNA binding domain comprising the amino acid sequence set forth in SEQ ID NO: 8. The truncated PB transposase domain may be attached to the MosI DNA binding domain via a linker sequence, resulting in the chimeric PB:MosI S544-58 transposase comprising the amino acid sequence set forth in SEQ ID NO: 9, with residues 3-58 of the MosI transposase highlighted in bold font, the PB domain underlined, and the linker sequence highlighted in italics:

(SEQ ID NO: 9)
MAPKKKRKVGGGGSSLDDEHILSALLQSDDELVGEDSDSEVSDHVSEDDV
QSDTEEAFIDEVHEVQPTSSGSEILDEQNVIEQPGSSLASNRILTLPQRT
IRGKNKHCWSTSKSTRRSRVSALNIVRSQRGPTRMCRNIYDPLLCFKLFF
TDEIISEIVKWTNAEISLKRRESMTSATFRDTNEDEIYAFFGILVMTAVR
KDNHMSTDDLFDRSLSMVYVSVMSRDRFDFLIRCLRMDDKSIRPTLREND
VFTPVRKIWDLFIHQCIQNYTPGAHLTIDEQLLGFRGRCPFRVYIPNKPS
KYGIKILMMCDSGTKYMINGMPYLGRGTQTNGVPLGEYYVKELSKPVHGS
CRNITCDNWFTSIPLAKNLLQEPYKLTIVGTVRSNKREIPEVLKNSRSRP
VGTSMFCFDGPLTLVSYKPKPAKMVYLLSSCDEDASINESTGKPQMVMYY
NQTKGGVDTLDQMCSVMTCSRKTNRWPMALLYGMINIACINSFIIYSHNV
SSKGEKVQSRKKFMRNLYMSLTSSFMRKRLEAPTLKRYLRDNISNILPKE
VPGTSGGGGSSFVPNKEQTRTVLIFCFHLKKTAAESHRMLVEAFGEQVPT
VKTCERWFQRFKSGDF.

In some embodiments, a chimeric PB:MosI transpose comprising a CRD deletion described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 9. In some embodiments, a chimeric PB:MosI transpose comprising a CRD deletion described herein comprises the amino acid sequence set forth in SEQ ID NO: 9 with one, two, three, four of five conservative amino acid substitutions. In some embodiments a chimeric PB:MosI transpose comprising a CRD deletion described herein comprises the amino acid sequence set forth in SEQ ID NO: 9.

In some embodiments, the chimeric transposase comprising a truncated piggyBac transposase residues comprising the amino acid sequence set forth in SEQ ID NO: 3 is connected to the two MosI DNA binding domains comprising the sequence of. SEQ ID NO: 6. The truncated PB transposase domain may be attached to the MosI DNA binding domain via a linker sequence, resulting in the chimeric PB:MosI V552-111 transposase comprising the amino acid sequence set forth in SEQ ID NO: 10, with residues 3-11 of the Mos1 transposase highlighted in bold, the PB domain underlined and the linker sequence highlighted in italics:

(SEQ ID NO: 10)
MAPKKKRKVGGGGSSLDDEHILSALLQSDDELVGEDSDSEVSDHVSEDDV
QSDTEEAFIDEVHEVQPTSSGSEILDEQNVIEQPGSSLASNRILTLPQRT
IRGKNKHCWSTSKSTRRSRVSALNIVRSQRGPTRMCRNIYDPLLCFKLFF
TDEIISEIVKWTNAEISLKRRESMTSATFRDTNEDEIYAFFGILVMTAVR
KDNHMSTDDLFDRSLSMVYVSVMSRDRFDFLIRCLRMDDKSIRPTLREND
VFTPVRKIWDLFIHQCIQNYTPGAHLTIDEQLLGFRGRCPFRVYIPNKPS
KYGIKILMMCDSGTKYMINGMPYLGRGTQTNGVPLGEYYVKELSKPVHGS
CRNITCDNWFTSIPLAKNLLQEPYKLTIVGTVRSNKREIPEVLKNSRSRP
VGTSMFCFDGPLTLVSYKPKPAKMVYLLSSCDEDASINESTGKPQMVMYY
NQTKGGVDTLDQMCSVMTCSRKTNRWPMALLYGMINIACINSFIIYSHNV
SSKGEKVQSRKKFMRNLYMSLTSSFMRKRLEAPTLKRYLRDNISNILPKE
VPGTSDDSTEEPVGGGGSSFVPNKEQTRTVLIFCFHLKKTAAESHRMLVE
AFGEQVPTVKTCERWFQRFKSGDFDVDDKEHGKPPKRYEDAELQALLDED
DAQTQKQLAEQLEVSQQAVSNRLREMG.

In some embodiments, a chimeric PB:MosI transpose comprising a CRD deletion described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 10. In some embodiments, a chimeric PB:MosI transpose comprising a CRD deletion described herein comprises the amino acid sequence set forth in SEQ ID NO: 10 with one, two, three, four of five conservative amino acid substitutions. In some embodiments a chimeric PB:MosI transpose comprising a CRD deletion described herein comprises the amino acid sequence set forth in SEQ ID NO: 10.

In some embodiments, the chimeric transposase comprising a truncated piggyBac transposase domain comprising the amino acid sequence of SEQ ID NO: 4 is connected to one MosI DNA binding domain comprising the amino acid sequence set forth in SEQ ID NO: 8. The truncated PB transposase domain may be attached to the MosI DNA binding domain via a linker sequence, resulting in the chimeric PB:MosI V552-58 transposase comprising the amino acid sequence set forth in SEQ ID NO: 11, with residues 3-58 of the MosI transposase highlighted in bold font, the PB domain underlined, and the linker sequence highlighted in italics:

(SEQ ID NO: 11)
MAPKKKRKVGGGGSSLDDEHILSALLQSDDELVGEDSDSEVSDHVSEDDV
QSDTEEAFIDEVHEVQPTSSGSEILDEQNVIEQPGSSLASNRILTLPQRT
IRGKNKHCWSTSKSTRRSRVSALNIVRSQRGPTRMCRNIYDPLLCFKLFF
TDEIISEIVKWTNAEISLKRRESMTSATFRDTNEDEIYAFFGILVMTAVR
KDNHMSTDDLFDRSLSMVYVSVMSRDRFDFLIRCLRMDDKSIRPTLREND
VFTPVRKIWDLFIHQCIQNYTPGAHLTIDEQLLGFRGRCPFRVYIPNKPS
KYGIKILMMCDSGTKYMINGMPYLGRGTQTNGVPLGEYYVKELSKPVHGS
CRNITCDNWFTSIPLAKNLLQEPYKLTIVGTVRSNKREIPEVLKNSRSRP
VGTSMFCFDGPLTLVSYKPKPAKMVYLLSSCDEDASINESTGKPQMVMYY
NQTKGGVDTLDQMCSVMTCSRKTNRWPMALLYGMINIACINSFIIYSHNV
SSKGEKVQSRKKFMRNLYMSLTSSFMRKRLEAPTLKRYLRDNISNILPKE
VPGTSDDSTEEPVGGGGSSFVPNKEQTRTVLIFCFHLKKTAAESHRMLVE
AFGEQVPTVKTCERWFQRFKSGDF.

In some embodiments, a chimeric PB:MosI transpose comprising a CRD deletion described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 11. In some embodiments, a chimeric PB:MosI transpose comprising a CRD deletion described herein comprises the amino acid sequence set forth in SEQ ID NO: 11 with one, two, three, four of five conservative amino acid substitutions. In some embodiments a chimeric PB:MosI transpose comprising a CRD deletion described herein comprises the amino acid sequence set forth in SEQ ID NO: 11.

The chimeric transposases may be used to transpose cells using transposons comprising chimeric transposon inverted repeat (ITR) polynucleotides described herein.

### Chimeric Transposon Inverted Terminal Repeat (ITR) Polynucleotides

Also provided herein are chimeric transposon inverted terminal repeat (ITR) polynucleotides, comprising in the 5′ to 3′ direction: (i) a truncated piggyBac left ITR sequence lacking the CRD binding site and (ii) a MosI ITR sequence comprising binding sites for two MosI DNA binding domains.

in some embodiments, the piggyBac left end (LE) ITR sequence comprises the sequence set forth in SEQ ID NO: 12. In some embodiments, the 35 bp LE PB ITR sequence is truncated after position 16, deleting the 19 bp CRD binding site. In some embodiments, the resulting truncated PB LE ITR sequence lacking the CRD binding site comprises only nucleotides 1-16 (highlighted in bold) of the full-length PB LE ITR sequence set forth in SEQ ID NO: 12: CCCTAGAAAGATAGTCTGCGTAAAATTGACGCATG (SEQ ID NO: 12). In some embodiments, the PB LE ITR sequence lacking the CRD binding site comprises the sequence CCCTAGAAAGATAGTC (SEQ ID NO: 172).

In some embodiments, the MosI ITR sequence comprising binding sites for two MosI DNA binding domains comprises the sequence:

(SEQ ID NO: 13)
TGTACAAGTATGAAATGTCGTTT.

In some embodiments, the 23 bp MosI sequence (SEQ ID NO: 13) containing the binding sites for the two MosI DNA binding domains is fused in reverse orientation to the truncated PB LE ITR (SEQ ID NO: 172), resulting in the LE PiggyBac: MosI ITR sequence set forth in SEQ ID NO: 15: CCCTAGAAAGATAGTCAAACGACATTTCATACTTGTACA (SEQ ID NO: 15)

In some embodiments, a single nucleotide is deleted from the 3′-end of the 23 bp MosI sequence containing the binding sites for the two MosI DNA binding domains (i.e., from the sequence set forth in SEQ ID NO: 13), leaving only residues 1-22, i.e., the sequence set forth in SEQ ID NO: 14: TGTACAAGTATGAAATGTCGTT (SEQ ID NO: 14).
in some embodiments, the 1-22 nucleotide MosI ITR fragment is fused in reverse orientation to the truncated PB LE ITR sequence set forth in SEQ ID NO: 12, resulting in the LE PiggyBac: MosI ITR-1 sequence set forth in SEQ ID NO: 16:

(SEQ ID NO: 16)
CCCTAGAAAGATAGTCAACGACATTTCATACTTGTACA.

In some embodiments, the chimeric transposon ITR polynucleotides further comprise one or two additional nucleotides between the truncated piggyBac left ITR sequence and the MosI ITR sequence. In some embodiments, one nucleotide is added to the 3′-end of the MosI ITR sequence to create the LE PiggyBac: MosI ITR+1 sequence set forth in SEQ ID NO: 17: CCCTAGAAAGATAGTCTAAACGACATTTCATACTTGTACA (SEQ ID NO: 17).

Similarly, two nucleotides may be added to the 3′-end of the MosI ITR sequence to create the LE PiggyBac: MosI ITR+2 sequence set forth in SEQ ID NO: 18: CCCTAGAAAGATAGTCTGAAACGACATTTCATACTTGTACA (SEQ ID NO: 18).

In some embodiments, the piggyBac right end (RE) ITR sequence comprises the sequence set forth in SEQ ID NO: 65. In some embodiments, the 63 bp RE PB ITR sequence is truncated after position 16, deleting the 19 bp CRD binding site and the remaining ITR sequence. The truncated PB RE ITR sequence lacking the CRD binding site comprises only nucleotides 1-16 (highlighted in bold) of the full-length PB RE ITR set forth in SEQ ID NO: 65: CCCTAGAAAGATAATCATATTGTGACGTACGTTAAAGATAATCATGCGTAAAATTG ACGCATG (SEQ ID NO: 65). In some embodiments, the truncated PB RE ITR sequence lacking the CRD binding site comprises the sequence CCCTAGAAAGATAATC (SEQ ID NO: 173).

In some embodiments, the MosI ITR sequence comprising binding sites for two MosI DNA binding domains comprises the sequence: TGTACAAGTATGAAATGTCGTTT (SEQ ID NO: 13).

In some embodiments, the 23 bp MosI sequence (set forth in SEQ ID NO: 13) containing the binding sites for the two MosI DNA binding domains is fused in reverse orientation to the truncated PB RE ITR (SEQ ID NO: 173) to create the RE PB:MosI ITR sequence set forth in SEQ ID NO: 19:

(SEQ ID NO: 19)
CCCTAGAAAGATAATCAAACGACATTTCATACTTGTACA

In some embodiments, a single nucleotide is deleted from the 3′-end of the 23 bp MosI sequence containing the binding sites for the two MosI DNA binding domains (set forth in SEQ ID NO: 13) resulting in the sequence: TGTACAAGTATGAAATGTCGTT (SEQ ID NO: 14).

In some embodiments, the 1-22 nucleotide MosI ITR fragment (SEQ ID NO: 14) is fused in reverse orientation to the truncated PB RE ITR (SEQ ID NO: 173 to create the RE Piggy Bac: MosI ITR-1 set forth in SEQ ID NO: 20:34

(SEQ ID NO: 20)
CCCTAGAAAGATAATCAACGACATTTCATACTTGTACA.

In some embodiments, the polynucleotides further comprise one or two additional nucleotides between the truncated piggyBac right ITR sequence and the MosI ITR sequence. In some embodiments, one nucleotide is added to the 3′-end of the MosI ITR sequence to create the RE PB:MosI ITR+1 sequence set forth in SEQ ID NO: 21:

(SEQ ID NO: 21)
CCCTAGAAAGATAATCAAAACGACATTTCATACTTGTACA.

Similarly, two nucleotides may be added to the 3′-end of the MosI ITR sequence to create the RE PiggyBac: MosI ITR+2 sequence set forth in SEQ ID NO: 22:

(SEQ ID NO: 22)
CCCTAGAAAGATAATCATAAACGACATTTCATACTTGTACA

The chimeric transposon ITR polynucleotides may be incorporated into vectors or into transposons (as described below) for use in conjunction with the chimeric transposases, chimeric site-specific transposase fusion proteins and in the methods described herein.

### Chimeric Site-specific PB:MosI Transposase Fusion Proteins

Also provided herein are chimeric site-specific transposase fusion proteins comprising, in N-terminal to C-terminal order: (i) a target-specific DNA binding domain, (ii) a truncated, N-terminal deleted, integration-deficient piggyBac transposase comprising a C-terminal Cysteine Rich Domain (CRD) deletion and (iii) one or more MosI DNA binding domain(s). In some embodiments, the target-specific DNA binding domain is a TAL or a zinc finger motif (ZFM).

### N-Terminal Pb Transposase Deletions

N-terminal deleted PB transposase sequences and integration-defective N-terminal piggyBac transposase previously have been described in International Patent Application No. PCT/2022/22549 and No. PCT/US2022/077549, each of which is incorporated herein by reference in its entirety for examples of integration-deficient N-terminally deleted piggyBac transposases that may be used in the constructs and fusion proteins described herein. In some embodiments, the N-terminal deletion of the truncated PB transposase is a deletion of amino acid residues 1-85, 1-86, 1-87, 1-88, 1-89, 1-90, 1-91, 1-92, 1-93, 1-94, 1-95, 1-96, 1-97, 1-98, 1-99, 1-100, 1-101, 1-102, or 1-103 of SEQ ID NO: 1. In some embodiments, the N-terminal deletion of the truncated PB transposase is a deletion of amino acid residues 1-93 of SEQ ID NO: 1, or corresponding residues of SEQ ID NOs: 3 or 4.

### Integration Deficient Transposases

In some embodiments, the chimeric site-specific transposase fusion protein comprises a chimeric transposase that is integration deficient. An integration-deficient transposase domain is a transposase that can excise its corresponding transposon, but that integrates the excised transposon at a lower frequency than a corresponding wild type transposase. Examples of integration-deficient transposases are disclosed in U.S. Pat. Nos. 6,218,185; 6,962,810, 8,399,643 and International Patent Application Publication No. WO 2019/173636, each of which is incorporated herein by reference in its entirety for examples of integration-deficient transposase that may be used in the constructs described herein. A list of integration-deficient amino acid substitutions is disclosed in U.S. Pat. No. 10,041,077, which is incorporated herein by reference in its entirety for examples of integration-deficient transposase that may be used in the constructs described herein. A wildtype SPB may be rendered integration-deficient by introducing mutations, for example, K93A, R372A, K375A, R376A and/or D450N (relative to SEQ ID NOs: 1, 3 or 4). It is believed that the introduction of mutations R372A, K375A, R376A and D450N renders the transposase integration deficient, but retains the excision function.

Thus, in some embodiments, the chimeric site-specific transposase fusion protein comprises a chimeric transposase domain comprising the sequence of SEQ ID NO: 1 with one of the following mutations: R372A, K375A, R376A and D450N. In some embodiments, the chimeric site-specific transposase fusion protein comprises a chimeric transposase domain comprising the sequence of SEQ ID NO: 1 with two of the following mutations: R372A, K375A, R376A and D450N. In some embodiments, the chimeric site-specific transposase fusion protein comprises a chimeric transposase domain comprising the sequence of SEQ ID NO: 1 with three of the following mutations: R372A, K375A, R376A and D450N. In some embodiments, the chimeric site-specific transposase fusion protein comprises a chimeric transposase domain comprising the sequence of SEQ ID NO: 1 with the following mutations: R372A, K375A, R376A and D450N.

Thus, in some embodiments, the chimeric site-specific transposase fusion protein comprises a chimeric transposase domain comprising the sequence of SEQ ID NO: 3 with one of the following mutations: R372A, K375A, R376A and D450N. In some embodiments, the chimeric site-specific transposase fusion protein comprises a chimeric transposase domain comprising the sequence of SEQ ID NO: 3 with two of the following mutations: R372A, K375A, R376A and D450N. In some embodiments, the chimeric site-specific transposase fusion protein comprises a chimeric transposase domain comprising the sequence of SEQ ID NO: 3 with three of the following mutations: R372A, K375A, R376A and D450N. In some embodiments, the chimeric site-specific transposase fusion protein comprises a chimeric transposase domain comprising the sequence of SEQ ID NO: 3 with the following mutations: R372A, K375A, R376A and D450N.

Thus, in some embodiments, the chimeric site-specific transposase fusion protein comprises a chimeric transposase domain comprising the sequence of SEQ ID NO: 4 with one of the following mutations: R372A, K375A, R376A and D450N. In some embodiments, the chimeric site-specific transposase fusion protein comprises a chimeric transposase domain comprising the sequence of SEQ ID NO: 4 with two of the following mutations: R372A, K375A, R376A and D450N. In some embodiments, the chimeric site-specific transposase fusion protein comprises a chimeric transposase domain comprising the sequence of SEQ ID NO: 4 with three of the following mutations: R372A, K375A, R376A and D450N. In some embodiments, the chimeric site-specific transposase fusion protein comprises a chimeric transposase domain comprising the sequence of SEQ ID NO: 4 with the following mutations: R372A, K375A, R376A and D450N.

An illustrative sequence of a truncated, N-terminal deleted, integration-deficient transposase comprises a deletion of amino acids 1-93 of the N-terminus of the PB transposase sequence and comprising three integration-deficient mutations is shown in SEQ ID NO: 85:

(SEQ ID NO: 85)
NKHCWSTSKSTRRSRVSALNIVRSQRGPTRMCRNIYDPLLCFKL
FFTDEIISEIVKWTNAEISLKRRESMTSATFRDTNEDEIYAFFG
ILVMTAVRKDNHMSTDDLFDRSLSMVYVSVMSRDRFDFLIRCLR
MDDKSIRPTLRENDVFTPVRKIWDLFIHQCIQNYTPGAHLTIDE
QLLGFRGRCPFRVYIPNKPSKYGIKILMMCDSGTKYMINGMPYL
GRGTQTNGVPLGEYYVKELSKPVHGSCRNITCDNWFTSIPLAKN
LLQEPYKLTIVGTVASNAREIPEVLKNSRSRPVGTSMFCFDGPL
TLVSYKPKPAKMVYLLSSCDEDASINESTGKPQMVMYYNQTKGG
VDTLNQMCSVMTCSRKTNRWPMALLYGMINIACINSFIIYSHNV
SSKGEKVQSRKKFMRNLYMSLTSSFMRKRLEAPTLKRYLRDNIS
NILPKEVPGTSDDSTEEPVMKKRTYCTYCPSKIRRKANASCKKC
KKVICREHNIDMCQSCF.

In some embodiments, a truncated, N-terminal deleted, integration-deficient transposase described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 85. In some embodiments, a truncated, N-terminal deleted, integration-deficient transposase described herein comprises the amino acid sequence set forth in SEQ ID NO: 85 with one, two, three, four of five conservative amino acid substitutions. In some embodiments a truncated, N-terminal deleted, integration-deficient transposase described herein comprises the amino acid sequence set forth in SEQ ID NO: 85. In some embodiments, a truncated, N-terminal deleted, integration-deficient transposase described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 89. In some embodiments, a truncated, N-terminal deleted, integration-deficient transposase described herein comprises the amino acid sequence set forth in SEQ ID NO: 89 with one, two, three, four of five conservative amino acid substitutions. In some embodiments, a truncated, N-terminal deleted, integration-deficient transposase described herein comprises the amino acid sequence set forth in SEQ ID NO: 89. In some embodiments, a truncated, N-terminal deleted, integration-deficient transposase described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 97. In some embodiments, a truncated, N-terminal deleted, integration-deficient transposase described herein comprises the amino acid sequence set forth in SEQ ID NO: 97 with one, two, three, four of five conservative amino acid substitutions. In some embodiments, a truncated, N-terminal deleted, integration-deficient transposase described herein comprises the amino acid sequence set forth in SEQ ID NO: 97. In some embodiments, a truncated, N-terminal deleted, integration-deficient transposase described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 110. In some embodiments, a truncated, N-terminal deleted, integration-deficient transposase described herein comprises the amino acid sequence set forth in SEQ ID NO: 110 with one, two, three, four of five conservative amino acid substitutions. In some embodiments, a truncated, N-terminal deleted, integration-deficient transposase described herein comprises the amino acid sequence set forth in SEQ ID NO: 110. In some embodiments, a truncated, N-terminal deleted, integration-deficient transposase described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 118. In some embodiments, a truncated, N-terminal deleted, integration-deficient transposase described herein comprises the amino acid sequence set forth in SEQ ID NO: 118 with one, two, three, four of five conservative amino acid substitutions. In some embodiments, a truncated, N-terminal deleted, integration-deficient transposase described herein comprises the amino acid sequence set forth in SEQ ID NO: 118. In some embodiments, a truncated, N-terminal deleted, integration-deficient transposase described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 131. In some embodiments, a truncated, N-terminal deleted, integration-deficient transposase described herein comprises the amino acid sequence set forth in SEQ ID NO: 131 with one, two, three, four of five conservative amino acid substitutions. In some embodiments, a truncated, N-terminal deleted, integration-deficient transposase described herein comprises the amino acid sequence set forth in SEQ ID NO: 131. In some embodiments, a truncated, N-terminal deleted, integration-deficient transposase described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 139. In some embodiments, a truncated, N-terminal deleted, integration-deficient transposase described herein comprises the amino acid sequence set forth in SEQ ID NO: 139 with one, two, three, four of five conservative amino acid substitutions. In some embodiments, a truncated, N-terminal deleted, integration-deficient transposase described herein comprises the amino acid sequence set forth in SEQ ID NO: 139. In some embodiments, a truncated, N-terminal deleted, integration-deficient transposase described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 152. In some embodiments, a truncated, N-terminal deleted, integration-deficient transposase described herein comprises the amino acid sequence set forth in SEQ ID NO: 152 with one, two, three, four of five conservative amino acid substitutions. In some embodiments, a truncated, N-terminal deleted, integration-deficient transposase described herein comprises the amino acid sequence set forth in SEQ ID NO: 152. In some embodiments, a truncated, N-terminal deleted, integration-deficient transposase described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 160. In some embodiments, a truncated, N-terminal deleted, integration-deficient transposase described herein comprises the amino acid sequence set forth in SEQ ID NO: 160 with one, two, three, four of five conservative amino acid substitutions. In some embodiments, a truncated, N-terminal deleted, integration-deficient transposase described herein comprises the amino acid sequence set forth in SEQ ID NO: 160.

In some embodiments, a truncated, N-terminal deleted, integration-deficient transposase described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in any one of SEQ ID NOs: 87-170. In some embodiments, a truncated, N-terminal deleted, integration-deficient transposase described herein comprises the amino acid sequence set forth in any one of SEQ ID NOs: 87-170 with one, two, three, four of five conservative amino acid substitutions. In some embodiments a truncated, N-terminal deleted, integration-deficient transposase described herein comprises the amino acid sequence set forth in any one of SEQ ID NOs: 87-170.

### DNA Targeting Domains

The chimeric site-specific transposase fusion proteins of the present disclosure may further comprise one or more DNA targeting domains. A DNA-targeting domain may be attached to the C-terminus or the N-terminus of the chimeric site-specific transposase fusion protein. In preferred embodiments, the DNA-targeting domain is attached to the N-terminus of the chimeric site-specific transposase fusion protein. Without wishing to be bound by theory, it is believed that addition of a DNA targeting domain to a chimeric site-specific transposase fusion protein improves site-specific transposase activity by targeting the chimeric site-specific transposase fusion protein fused to the DNA targeting domain to the targeted site. In some embodiments, the insertion of a DNA targeting domain improves site-specific transposase activity by at least 2-fold, at least 3-fold, at least 4-fold, or at least 5-fold compared to the same chimeric site-specific transposase not comprising a DNA targeting domain.

Any DNA targeting domain known in the art may be used in the context of the chimeric transposases and chimeric site-specific transposase fusion proteins provided herein described herein, including, without limitation, CRISPR, Zinc Finger Motifs, TALE, and transcription factors.

Methods of engineering Zinc-Finger Nucleases that bind to specific targets are described in, for example, Sander et al., Nat Methods. 2011 January; 8 (1): 67-69. In some embodiments, the DNA targeting domain comprises three Zinc Finger Motifs. In some embodiments, the three Zinc Finger Motifs are flanked by GGGGS (SEQ ID NO: 86) linkers. In some embodiments, the three Zinc Finger Motifs (ZFM) and flanking GGGGS (SEQ ID NO: 86) linkers cumulatively comprise the sequence set forth in SEQ ID NO: 23:

(SEQ ID NO: 23)
GGGGSERPYACPVESCDRRFSRSDELTRHIRIHTGQKPFQCRIC
MRNFSRSDHLTTHIRTHTGEKPFACDICGRKFARSDERKRHTKI
HLRQKDGGGGS

or a sequence having at least 75%, at least 80%, at least 85%, at least 90%, at least 95%, at least 98%, or at least 99% identity thereto.

In some embodiments, provided herein is a chimeric site-specific transposase fusion protein comprising three ZFM domains joined through a linker sequence to a chimeric PBx: MosI transposase of the present disclosure. An illustrative chimeric ZFM-PB:MosI transposase fusion protein comprises three ZFM domains joined to a chimeric S544:PB:MosI-58 transposase to create a ZFM-S544:PB:MosI-58 chimeric site-specific transposase comprising the nucleic acid sequence:

(SEQ ID NO: 24)
MAPKKKRKVGGGGSERPYACPVESCDRRFSRSDELTRHIRIHTG
QKPFQCRICMRNFSRSDHLTTHIRTHTGEKPFACDICGRKFARS
DERKRHTKIHLRQKDGGGGSNKHCWSTSKSTRRSRVSALNIVRS
QRGPTRMCRNIYDPLLCFKLFFTDEIISEIVKWTNAEISLKRRE
SMTSATFRDTNEDEIYAFFGILVMTAVRKDNHMSTDDLFDRSLS
MVYVSVMSRDRFDFLIRCLRMDDKSIRPTLRENDVFTPVRKIWD
LFIHQCIQNYTPGAHLTIDEQLLGFRGRCPFRVYIPNKPSKYGI
KILMMCDSGTKYMINGMPYLGRGTQTNGVPLGEYYVKELSKPVH
GSCRNITCDNWFTSIPLAKNLLQEPYKLTIVGTVASNAREIPEV
LKNSRSRPVGTSMFCFDGPLTLVSYKPKPAKMVYLLSSCDEDAS
INESTGKPQMVMYYNQTKGGVDTLNQMCSVMTCSRKTNRWPMAL
LYGMINIACINSFIIYSHNVSSKGEKVQSRKKFMRNLYMSLTSS
FMRKRLEAPTLKRYLRDNISNILPKEVPGTSGGGGSSFVPNKEQ
TRTVLIFCFHLKKTAAESHRMLVEAFGEQVPTVKTCERWFQRFK
SGDF

In some embodiments, a chimeric ZFM-PB:MosI transposase fusion protein described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 24. In some embodiments, a chimeric ZFM-PB:MosI transposase fusion protein described herein comprises the amino acid sequence set forth in SEQ ID NO: 24 with one, two, three, four of five conservative amino acid substitutions. In some embodiments a chimeric ZFM-PB:MosI transposase fusion protein described herein comprises the amino acid sequence set forth in SEQ ID NO: 24.

Another illustrative chimeric ZFM-PB:MosI transposase fusion protein comprises three ZFM domains joined to a chimeric S544 PB:MosI-111 transposase to create a ZFM-S544:PB:MosI-111 chimeric site-specific transposase comprising the amino acid sequence:

(SEQ ID NO: 25)
MAPKKKRKVGGGGSERPYACPVESCDRRFSRSDELTRHIRIHTG
QKPFQCRICMRNFSRSDHLTTHIRTHTGEKPFACDICGRKFARS
DERKRHTKIHLRQKDGGGGSNKHCWSTSKSTRRSRVSALNIVRS
QRGPTRMCRNIYDPLLCFKLFFTDEIISEIVKWTNAEISLKRRE
SMTSATFRDTNEDEIYAFFGILVMTAVRKDNHMSTDDLFDRSLS
MVYVSVMSRDRFDFLIRCLRMDDKSIRPTLRENDVFTPVRKIWD
LFIHQCIQNYTPGAHLTIDEQLLGFRGRCPFRVYIPNKPSKYGI
KILMMCDSGTKYMINGMPYLGRGTQTNGVPLGEYYVKELSKPVH
GSCRNITCDNWFTSIPLAKNLLQEPYKLTIVGTVASNAREIPEV
LKNSRSRPVGTSMFCFDGPLTLVSYKPKPAKMVYLLSSCDEDAS
INESTGKPQMVMYYNQTKGGVDTLNQMCSVMTCSRKTNRWPMAL
LYGMINIACINSFIIYSHNVSSKGEKVQSRKKFMRNLYMSLTSS
FMRKRLEAPTLKRYLRDNISNILPKEVPGTSGGGGSSFVPNKEQ
TRTVLIFCFHLKKTAAESHRMLVEAFGEQVPTVKTCERWFQRFK
SGDFDVDDKEHGKPPKRYEDAELQALLDEDDAQTQKQLAEQLEV
SQQAVSNRLREMG

In some embodiments, a chimeric ZFM-PB:MosI transposase fusion protein described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 25. In some embodiments, a chimeric ZFM-PB:MosI transposase fusion protein described herein comprises the amino acid sequence set forth in SEQ ID NO: 25 with one, two, three, four of five conservative amino acid substitutions. In some embodiments a chimeric ZFM-PB:MosI transposase fusion protein described herein comprises the amino acid sequence set forth in SEQ ID NO: 25.

Yet another illustrative chimeric ZFM-PB:MosI transposase fusion protein comprises three ZFM domains joined to a chimeric V552 PB:MosI-58 transposase to create a ZFM-PB:MosI-58 chimeric site-specific transposase comprising the nucleic acid sequence:

(SEQ ID NO: 26)
MAPKKKRKVGGGGSERPYACPVESCDRRFSRSDELTRHIRIHTG
QKPFQCRICMRNFSRSDHLTTHIRTHTGEKPFACDICGRKFARS
DERKRHTKIHLRQKDGGGGSNKHCWSTSKSTRRSRVSALNIVRS
QRGPTRMCRNIYDPLLCFKLFFTDEIISEIVKWTNAEISLKRRE
SMTSATFRDTNEDEIYAFFGILVMTAVRKDNHMSTDDLFDRSLS
MVYVSVMSRDRFDFLIRCLRMDDKSIRPTLRENDVFTPVRKIWD
LFIHQCIQNYTPGAHLTIDEQLLGFRGRCPFRVYIPNKPSKYGI
KILMMCDSGTKYMINGMPYLGRGTQTNGVPLGEYYVKELSKPVH
GSCRNITCDNWFTSIPLAKNLLQEPYKLTIVGTVASNAREIPEV
LKNSRSRPVGTSMFCFDGPLTLVSYKPKPAKMVYLLSSCDEDAS
INESTGKPQMVMYYNQTKGGVDTLNQMCSVMTCSRKTNRWPMAL
LYGMINIACINSFIIYSHNVSSKGEKVQSRKKFMRNLYMSLTSS
FMRKRLEAPTLKRYLRDNISNILPKEVPGTSDDSTEEPVGGGGS
SFVPNKEQTRTVLIFCFHLKKTAAESHRMLVEAFGEQVPTVKTC
ERWFQRFKSGDF.

In some embodiments, a chimeric ZFM-PB:MosI transposase fusion protein described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 26. In some embodiments, a chimeric ZFM-PB:MosI transposase fusion protein described herein comprises the amino acid sequence set forth in SEQ ID NO: 26 with one, two, three, four of five conservative amino acid substitutions. In some embodiments a chimeric ZFM-PB:MosI transposase fusion protein described herein comprises the amino acid sequence set forth in SEQ ID NO: 26.

Another illustrative chimeric ZFM-PB:MosI transposase fusion protein comprises three ZFM domains joined to a chimeric V552:PB:MosI-111 transposase to create a ZFM-V552:PB:MosI-111 chimeric site-specific transposase comprising the nucleic acid sequence:

(SEQ ID NO: 27)
MAPKKKRKVGGGGSERPYACPVESCDRRFSRSDELTRHIRIHTG
QKPFQCRICMRNFSRSDHLTTHIRTHTGEKPFACDICGRKFARS
DERKRHTKIHLRQKDGGGGSNKHCWSTSKSTRRSRVSALNIVRS
QRGPTRMCRNIYDPLLCFKLFFTDEIISEIVKWTNAEISLKRRE
SMTSATFRDTNEDEIYAFFGILVMTAVRKDNHMSTDDLFDRSLS
MVYVSVMSRDRFDFLIRCLRMDDKSIRPTLRENDVFTPVRKIWD
LFIHQCIQNYTPGAHLTIDEQLLGFRGRCPFRVYIPNKPSKYGI
KILMMCDSGTKYMINGMPYLGRGTQTNGVPLGEYYVKELSKPVH
GSCRNITCDNWFTSIPLAKNLLQEPYKLTIVGTVASNAREIPEV
LKNSRSRPVGTSMFCFDGPLTLVSYKPKPAKMVYLLSSCDEDAS
INESTGKPQMVMYYNQTKGGVDTLNQMCSVMTCSRKTNRWPMAL
LYGMINIACINSFIIYSHNVSSKGEKVQSRKKFMRNLYMSLTSS
FMRKRLEAPTLKRYLRDNISNILPKEVPGTSDDSTEEPVGGGGS
SFVPNKEQTRTVLIFCFHLKKTAAESHRMLVEAFGEQVPTVKTC
ERWFQRFKSGDFDVDDKEHGKPPKRYEDAELQALLDEDDAQTQK
QLAEQLEVSQQAVSNRLREMG.

In some embodiments, a chimeric ZFM-PB:MosI transposase fusion protein described herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 27. In some embodiments, a chimeric ZFM-PB:MosI transposase fusion protein described herein comprises the amino acid sequence set forth in SEQ ID NO: 27 with one, two, three, four of five conservative amino acid substitutions. In some embodiments a chimeric ZFM-PB:MosI transposase fusion protein described herein comprises the amino acid sequence set forth in SEQ ID NO: 27.

In some aspects, the DNA targeting domain is a TAL array. TALEs (Transcription activator-like effectors) from Xanthomonas typically contain a 288 amino acid N-terminus followed by an array of a variable number of about 34 amino acid long repeating sequences (repeats) followed by a 278 amino acid C-terminus (SEQ ID NO: 29); however, truncated versions have been described in the literature (e.g., see Miller et al., Nat Biotechnol 29, 143-148 (2011). TALs fused to a FokI nuclease (a TAL effector nuclease; TALEN) usually contain truncations of the N and C termini. For example, the first 152 amino acids of the N-terminus may be removed (called delta 152; the sequence of the N-terminus after removal of the first 152 amino acids is set forth in SEQ ID No: 30) and the C-terminus may be truncated leaving 63 amino acids (called +63; the sequence is set forth in SEQ ID NO: 31).

TALs generally contain arrays of 34 amino acids repeated a variable number of times. The two amino acids at position 12 and 13 are usually varied and determine which nucleotide the TAL repeat will recognize. Thus, the amino acids NG recognize T, NI recognize A, NN recognize G or A, HD recognize C, NK recognize G, NS recognize A, C, G or T. Other amino acids within the 34 residue repeat may also be varied. For example position 11 is often changed to an N for repeats that recognize G. Also, positions 4 and 32 are often varied to reduce the repetitiveness of the array, but not to determine the binding specificity. The number of 34 amino acid repeats in an array determines the length of the DNA sequence recognized (one protein repeat binds one DNA base). Furthermore, the last base is recognized by a “half array” that is 20 amino acids rather than 34. This feature allows a TAL array to be programed to bind a specific DNA sequence. A person of skill in the art will be able to modify the TALEN sequences to achieve the desired target specificity.

In addition, an N-terminal domain of TALs (e.g., an N-terminal domain comprising the sequence set forth in SEQ ID NO: 30) recognizes, and requires for binding, a T that is located immediately 5′ of the target DNA sequence. Mutations of TAL N-terminal domains that no longer require a 5′ T have been described in the literature (see, e.g., Lamb et al., Nucleic Acids Res. 2013 November; 41 (21): 9779-85) For example, the NT-G mutant comprising the amino acid sequence set forth in SEQ ID NO: 32 requires a 5′G instead of a 5′T, while the NT-BN mutant comprising the amino acid sequence set forth in SEQ ID NO: 33 does not require any specific 5′ nucleotide. These mutated N-terminal domain sequences may be used to provide additional options to develop sequences of fusion proteins described herein that may be targeted using TAL Arrays.

In some embodiments, a TAL array provided herein comprises nine 34-amino acid repeats followed by the 20-amino acid “half” repeat with flanking BsmBI type IIS restriction sites. In one embodiment, individual TAL modules containing 34-amino acid repeats or a 20-amino acid “half” repeat may be designed and synthesized flanked by BsmBI type IIS restriction sites. In some embodiments, the entire TAL module set contains 4 modules capable of recognizing either A, C, G, T for each of 10 bp positions (i.e., 40 TAL modules for 10 bp recognized target), and one TAL half repeat module. Illustrative TAL modules are set forth in SEQ ID NOs: 34-37, wherein X is any amino acid:

TAL Module Version 1:
(SEQ ID NO: 34)
LTPDQVVAIAXXXGGKQALETVQRLLPVLCQDHG
TAL Module Version 2:
(SEQ ID NO: 35)
LTPEQVVAIAXXXGGKQALETVQRLLPVLCQAHG
TAL Module Version 3:
(SEQ ID NO: 36)
LTPDQVVAIAXXXGGKQALETVQRLLPVLCQAHG
TAL Module Version 4:
(SEQ ID NO: 37)
LTPAQVVAIAXXXGGKQALETVQRLLPVLCQDHG.

An illustrative TAL Half Module is set forth in SEQ ID NO: 38, wherein X is any amino acid: LTPEQVVAIAXXXGGRPALE (SEQ ID NO: 38).

Pairs of TAL arrays targeting sequences in the desired gene may be designed and the corresponding modules selected and pooled together using “Golden Gate Assembly,” to assemble in frame each TAL-Array. The DNA sequence encoding TAL Arrays generated herein may be further codon optimized using GeneArt algorithms (Thermo Fisher™).

TAL arrays may be used to target TTAA sites in the genome. When designing left and right TAL Arrays comprising a N-terminal domain recognizing a T and a TAL C-terminal domain to be fused to a chimeric site-specific transposase, one TAL Array recognizes a sequence 5′ of the sequence TTAA, and the other TAL Array recognizes a sequence 3′ of the sequence TTAA. Since the sequence 5′ of TTAA is most often different from the sequence 3′ of TTAA in genomic DNA targets, TAL-site-specific Super piggyBac transposase fusion proteins (“TAL-ssSPB” or “TAL-PBx”) will most often be used as a heterodimer consisting of two transposase fusion proteins, each comprising a different TAL domain, which recognize two different DNA sequences. Additionally, the sequence recognized by the TAL Array is not necessarily directly adjacent to the TTAA. Instead, it may be separated from the TTAA by a spacer of a given bp length, e.g., spacers of 12 bp, 13 bp or 14 bp.

A TAL array may target any DNA sequence (e.g., genomic DNA sequence) of interest. It will be apparent to a person of skill in the art that any left TAL array for a given target can be combined with any right TAL array for the same target.

In some embodiments, a TAL array targets green fluorescent protein (GFP). TAL-piggyBac transposase fusion proteins comprising N-terminal deleted piggyBac transposase sequences and integration defective N-terminal piggyBac transposase targeting GFP have been described in co-owned International Patent Application No. PCT/2022/22549, which is incorporated herein in its entirety for examples of TAL-piggyBac transposase fusion proteins that may be used in the constructs described herein.

Illustrative sequences of right TAL arrays that may be incorporated into chimeric site-specific transposase fusion proteins targeting GFP are set forth in SEQ ID NOs: 41-44.

In some embodiments, a TAL array targets a LINE1 repeat element. TAL-piggyBac transposase fusion proteins comprising N-terminal deleted piggyBac transposase sequences and integration defective N-terminal piggyBac transposase targeting LINE1 elements have been described in International Patent Application No. PCT/2022/22549, which is incorporated herein in its entirety for examples of TAL-piggyBac transposase fusion proteins that may be used in the constructs described herein.

Illustrative sequences of left and right TAL arrays targeting LINE1 are set forth in SEQ ID NOs: 66 and 67, respectively.

### Nuclear Localization Signals

In some embodiments, the chimeric transposases and chimeric site-specific transposase fusion proteins provided herein may comprise an in-frame nuclear localization sequence (NLS). Examples of transposases fused to a nuclear localization signal are disclosed in U.S. Pat. Nos. 6,218,185; 6,962,810, 8,399,643 and International Patent Application Publication No. WO 2019/173636, each of which is incorporated herein in its entirety for examples of transposases fused to an NLS that may be used in the constructs described herein. In some embodiments, the NLS comprises the sequence of PKKKRKV (SEQ ID NO: 39). In certain aspects, the in-frame NLS is located upstream (N-terminal) of the transposase domain comprising an N-terminal deletion. In certain aspects, the in-frame NLS is located downstream (C-terminal) of the transposase domain comprising an N-terminal deletion.

In general, the NLS is preferably located at the N-terminal end of a chimeric transposase or chimeric site-specific transposase fusion protein.

In certain aspects, the in-frame NLS is fused directly to the amino terminus of a chimeric transposase or a chimeric site-specific transposase fusion protein. In some embodiments, an initiator methionine is introduced before the NLS. In some embodiments, additional adenine residues are introduced before and/or after the NLS to ensure in-frame translation. As such, the numbering of the residues in SEQ ID NO: 5 begins at the 12th residue of SEQ ID NO: 5 for the purpose of identifying deleted and mutated residues.

### Nucleic Acids

Also provided herein are polynucleotides comprising nucleic acid sequences encoding the chimeric transposases and chimeric site-specific transposase fusion proteins described herein. In some embodiments, the polynucleotides are isolated or purified.

The isolated polynucleotides of the disclosure can be made using (a) recombinant methods, (b) synthetic techniques, (c) purification techniques, and/or (d) combinations thereof, which are well-known in the art. Methods of constructing nucleic acids encoding the chimeric transposases and chimeric site-specific transposase fusion proteins described herein are well known in the art or described herein, for example, PCR-based mutagenesis.

The fusion of the present invention can be generated using any suitable method known in the art or described herein.

The isolated polynucleotides of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library.

Methods of amplification of RNA or DNA are well known in the art and can be used according to the disclosure without undue experimentation, based on the teaching and guidance presented herein. Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA-mediated amplification that uses anti-sense RNA to the target sequence as a template for double-stranded DNA synthesis (U.S. Pat. No. 5,130,238 to Malek, et al, with the tradename NASBA), the contents of each of which is incorporated herein by reference.

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech™). Additionally, e.g., the T4 gene 32 protein (Boehringer Mannheim) can be used to improve yield of long PCR products.

The polynucleotides of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Expression Vectors and Host Cells

The disclosure also relates to vectors that include polynucleotides of the disclosure, host cells that are genetically engineered with the recombinant vectors, and the production of at least one protein scaffold by recombinant techniques, as is well known in the art. See, e.g., Sambrook, et al., supra; Ausubel, et al., supra, each incorporated herein by reference in its entirety.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

In some embodiments, the DNA insert is operatively linked to an appropriate promoter. In some embodiments, the promoter is an EF-la promoter comprising the sequence set forth in SEQ ID NO: 45. The expression constructs may further contain sites for transcription initiation, termination and, in the transcribed region, a ribosome binding site for translation. The coding portion of the mature transcripts expressed by the constructs will preferably include a translation start codon (e.g., ATG) at the beginning and a termination codon (e.g., UAA, UGA or UAG) appropriately positioned at the end of the mRNA to be translated, with UAA and UAG preferred for mammalian or eukaryotic cell expression.

Expression vectors may further comprise one or more selectable markers. Examples of selectable markers include, but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotes. Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors may comprise at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. In some embodiments, the selectable cell surface marker distinguishes those cells modified by a composition or method of the disclosure from those cells that are not modified by a composition or method of the disclosure. Examples of cell surface markers include, but are not limited to, “cluster of designation” or “classification determinant” proteins (often abbreviated as “CD”) such as a truncated or full-length form of CD19, CD271, CD34, CD22, CD20, CD33, CD52, or any combination thereof. Cell surface markers further include the suicide gene marker RQR8 (Philip B et al. Blood. 2014 Aug. 21; 124 (8): 1277-87).

Expression vectors may further comprise at least one selectable drug resistance marker for isolation of cells modified by the compositions and methods of the disclosure. Selectable drug resistance markers of the disclosure may comprise wild-type or mutant Neo, DHFR, TYMS, FRANCE, RAD51C, GCS, MDR1, ALDH1, NKX2.2, or any combination thereof.

Those of ordinary skill in the art are knowledgeable in the numerous expression systems available for expression of a nucleic acid encoding a protein of the disclosure. Nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, each of which is incorporated herein by reference in its entirety.

Cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a preferred aspect, the recombinant cell is a P3X63Ab8.653 or an SP2/0-Ag14 cell.

Expression vectors for these cells can include one or more of the following expression control sequences: An origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491)), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present disclosure are known and available, for instance, from the American Type Culture Collection Catalogue of Cell Lines and Hybridomas (www.atcc.org) or other known or commercial sources.

When eukaryotic host cells are employed, polyadenylation or transcription terminator sequences are typically incorporated into the vector. An example of a terminator sequence is the polyadenylation sequence from the bovine growth hormone gene. In some embodiments, the polyA sequence is an SV40 polyA sequence comprising the sequence set forth in SEQ ID NO: 47.

Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

The plasmid constructs described herein may be used to deliver nucleic acids encoding the transposase domains or fusion proteins described herein to a cell.

The transposase domains and fusion proteins described herein may also be delivered to a cell using mRNA constructs. Thus, in one embodiment, provided herein is an mRNA sequence encoding a transposase domain or a fusion protein described herein. Such mRNA sequences may be delivered to a cell using a nanoparticle, for example, a lipid nanoparticle. Examples of lipid nanoparticles are described in, e.g., International Patent Application Publications No. WO 2022/087148, No. WO 2022/182792, No. WO 2023/141576, No. WO 2024/035783, and No. WO 2023/141576, each of which is incorporated herein by reference in its entirety for examples of lipid nanoparticles that may be used to deliver mRNA constructs encoding the fusion proteins or transposase domains described herein. An mRNA construct may also be delivered to a cell by electroporation or nucleofection. The mRNA may be capped or otherwise modified.

### Transposons, Cells and Modified Cells

The chimeric transposases and chimeric site-specific transposase fusion proteins described herein may be used in conjunction with a transposon to modify cells. The transposon can be a piggyBac™ (PB) transposon. In some embodiments, when the transposon is a PB transposon, the transposase is a piggyBac™ (PB) transposase, a piggyBac-like (PBL) transposase or a Super piggyBac™ (SPB) transposase. Non-limiting examples of PB transposons are described in detail in U.S. Pat. Nos. 6,218,182; 6,962,810; 8,399,643 and PCT Publication No. WO 2010/099296, each of which is incorporated herein by reference in its entirety for examples of transposons that may be used in the methods disclosed herein. The transposons can comprise a nucleic acid encoding a therapeutic protein or therapeutic agent. Examples of therapeutic proteins include those disclosed in PCT Publication No. WO 2019/173636 and No. WO 2020/051374, each of which is incorporated herein by reference in its entirety for examples of therapeutic proteins that may be used in the chimeric transposases and chimeric site-specific transposase fusion proteins described herein.

In some embodiments, the transposon may be a transposon comprising one or more chimeric PB:MosI ITR polynucleotides described herein. In some embodiments, the transposon comprises a chimeric LE PB: Mos1 ITR polynucleotide and a chimeric RE PB:MosI ITR polynucleotide. In some embodiments, the LE PB:MosI ITR polynucleotide is PB:MosI ITR comprising the sequence set forth in SEQ ID NO: 19. In some embodiments, the LE PB:MosI ITR polynucleotide is PB:MosI ITR-1 comprising the sequence set forth in SEQ ID NO: 20. In some embodiments, the LE PB:MosI ITR polynucleotide is PB:MosI ITR+1 comprising the sequence of SEQ ID NO: 21. In some embodiments, the LE PB:MosI ITR polynucleotide is PB:MosI ITR+2 comprising the sequence of SEQ ID NO: 22.

In some embodiments, the RE PB:MosI ITR polynucleotide is PB:MosI ITR comprising the sequence of SEQ ID NO: 15. In some embodiments, the RE PB:MosI ITR polynucleotide is PB:MosI ITR-1 comprising the sequence of SEQ ID NO: 16. In some embodiments, the RE PB:MosI ITR polynucleotide is PB:MosI ITR+1 comprising the sequence of SEQ ID NO: 17. In some embodiments, the RE PB:MosI ITR polynucleotide is PB:MosI ITR+2 comprising the sequence of SEQ ID NO: 18.

In some embodiments, the PB:MosI ITR transposon comprises the nucleotide sequence: TTAACCCTAGAAAGATAGTCAAACGACATTTCATACTTGTACAGACGCATGCATTCT TGAAATATTGCTCTCTCTTTCTAAATAGCGCGAATCCGTCGCTGTGCATTTAGGACAT CTCAGTCGCGAGCTGAAGGGCATCGACTTCAAGGAGGACGGCAACATCCTGGGGCA CAAGCTGGAGTACAACTACAACAGCCACAACGTCTATATCATGGCCGACAAGCAGA AGAACGGCATCAAGGTGAACTTCAAGATCCGCCACAACATCGAGGACGGCAGCGTG CAGCTCGCCGACCACTACCAGCAGAAATGTACAAGTATGAAATGTCGTTTGATTATC TTTCTAGGGTTAA (SEQ ID NO: 50). In some embodiments, a PB:MosI ITR transposon described herein comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 50.

In some embodiments, the PB:MosI ITR-1 transposon comprises the nucleotide sequence: TTAACCCTAGAAAGATAGTCAACGACATTTCATACTTGTACAGACGCATGCATTCTT GAAATATTGCTCTCTCTTTCTAAATAGCGCGAATCCGTCGCTGTGCATTTAGGACATC TCAGTCGCGAGCTGAAGGGCATCGACTTCAAGGAGGACGGCAACATCCTGGGGCAC AAGCTGGAGTACAACTACAACAGCCACAACGTCTATATCATGGCCGACAAGCAGAA GAACGGCATCAAGGTGAACTTCAAGATCCGCCACAACATCGAGGACGGCAGCGTGC AGCTCGCCGACCACTACCAGCAGAAATGTACAAGTATGAAATGTCGTTGATTATCTT TCTAGGGTTAA (SEQ ID NO: 51). In some embodiments, a PB:MosI ITR transposon described herein comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 51.

In some embodiments, the PB:MosI ITR+1 transposon comprises the nucleotide sequence: TTAACCCTAGAAAGATAGTCTAAACGACATTTCATACTTGTACAGACGCATGCATTC TTGAAATATTGCTCTCTCTTTCTAAATAGCGCGAATCCGTCGCTGTGCATTTAGGACA TCTCAGTCGCGAGCTGAAGGGCATCGACTTCAAGGAGGACGGCAACATCCTGGGGC ACAAGCTGGAGTACAACTACAACAGCCACAACGTCTATATCATGGCCGACAAGCAG AAGAACGGCATCAAGGTGAACTTCAAGATCCGCCACAACATCGAGGACGGCAGCGT GCAGCTCGCCGACCACTACCAGCAGAAATGTACAAGTATGAAATGTCGTTTTGATTA TCTTTCTAGGGTTAA (SEQ ID NO: 52). In some embodiments, a PB:MosI ITR transposon described herein comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 52.

In some embodiments, the PB:MosI ITR+2 transposon comprises the nucleotide sequence: TTAACCCTAGAAAGATAGTCTGAAACGACATTTCATACTTGTACAGACGCATGCATT CTTGAAATATTGCTCTCTCTTTCTAAATAGCGCGAATCCGTCGCTGTGCATTTAGGAC ATCTCAGTCGCGAGCTGAAGGGCATCGACTTCAAGGAGGACGGCAACATCCTGGGG CACAAGCTGGAGTACAACTACAACAGCCACAACGTCTATATCATGGCCGACAAGCA GAAGAACGGCATCAAGGTGAACTTCAAGATCCGCCACAACATCGAGGACGGCAGCG TGCAGCTCGCCGACCACTACCAGCAGAAATGTACAAGTATGAAATGTCGTTTATGAT TATCTTTCTAGGGTTAA (SEQ ID NO: 53). In some embodiments, a PB:MosI ITR transposon described herein comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 53.

In some embodiments, a transposon disclosed herein comprises a shortened PB:MosI ITR polynucleotide. In some embodiments, the left end PB:MosI shortened ITR comprises the nucleic acid sequence set forth in SEQ ID NO: 54. In some embodiments, the right end PB:MosI shortened ITR comprises the nucleic acid sequence set forth in SEQ ID NO: 55. In some embodiments, the transposons comprise a left end PB:MosI shortened ITR and a right end PB:MosI shortened ITR to generate a PB:MosI short transposon comprising the nucleic acid sequence set forth in SEQ ID NO: 56. In some embodiments, a PB:MosI short transposon comprises a sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 56.

In another aspect, provided herein are modified cells comprising one or more transposon and one or more chimeric transposase or chimeric site-specific transposase fusion protein described herein. Cells and modified cells of the disclosure can be mammalian cells. Preferably, the cells and modified cells are human cells.

A cell modified using a chimeric transposase or chimeric site-specific transposase fusion protein described herein can be a germline cell or a somatic cell. Cells and modified cells of the disclosure can be immune cells, e.g., lymphoid progenitor cells, natural killer (NK) cells, T lymphocytes (T-cell), stem memory T cells (T_{SCM} cells), central memory T cells (T_{CM}), stem cell-like T cells, B lymphocytes (B-cells), antigen presenting cells (APCs), cytokine induced killer (CIK) cells, myeloid progenitor cells, neutrophils, basophils, eosinophils, monocytes, macrophages, platelets, erythrocytes, red blood cells (RBCs), megakaryocytes or osteoclasts. The modified cell can be differentiated, undifferentiated, or immortalized. The modified undifferentiated cell can be a stem cell. The modified undifferentiated cell can be an induced pluripotent stem cell. The modified cell can be a hepatocyte, a T cell, a hematopoietic stem cell, a natural killer cell, a macrophage, a dendritic cell, a monocyte, a megakaryocyte, or an osteoclast. The modified cell can be modified while the cell is quiescent, in an activated state, resting, in interphase, in prophase, in metaphase, in anaphase, or in telophase. The modified cell can be fresh, cryopreserved, bulk, sorted into sub-populations, derived from whole blood, from leukapheresis, or from an immortalized cell line. A detailed description for isolating cells from a leukapheresis product or blood is disclosed in in PCT Publications No. WO 2019/173636 and No. WO 2020/051374, each of which is incorporated herein by reference in its entirety.

The methods of the disclosure can be used to modify or produce a population of modified T cells, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of the plurality of modified T cells in the population expresses one or more cell-surface marker(s) of a stem memory T cell (T_{SCM}) or a T_{SCM}-like cell; and wherein the one or more cell-surface marker(s) comprise CD45RA and CD62L. The cell-surface markers of a T_{SCM} or a T_{SCM}-like cell can also comprise one or more of CD62L, CD45RA, CD28, CCR7, CD127, CD45RO, CD95, CD95 and IL-2RB. The cell-surface markers of a T_{SCM} or a T_{SCM}-like cell can also comprise one or more of CD45RA, CD95, IL-2RB, CCR7, and CD62L.

The disclosure provides methods of expressing a CAR on the surface of a cell. The method comprises (a) obtaining a cell population; (b) contacting the cell population with a composition comprising a CAR or a sequence encoding the CAR under conditions sufficient to transfer the CAR or the sequence encoding the CAR across a cell membrane of at least one cell in the cell population, thereby generating a modified cell population; (c) culturing the modified cell population under conditions suitable for integration of the sequence encoding the CAR; and (d) expanding and/or selecting at least one cell from the modified cell population that express the CAR on the cell surface. A more detailed description of methods for expressing a CAR on the surface of a cell is disclosed in PCT Publication No. WO 2019/049816 and No. WO 2020/051374.

The present disclosure further provides a cell or a population of cells wherein the cell comprises a composition comprising (a) an inducible transgene construct, comprising a sequence encoding an inducible promoter and a sequence encoding a transgene, and (b) a receptor construct, comprising a sequence encoding a constitutive promoter and a sequence encoding an exogenous receptor, such as a CAR, wherein, upon integration of the construct of (a) and the construct of (b) into a genomic sequence of a cell, the exogenous receptor is expressed, and wherein the exogenous receptor, upon binding a ligand or antigen, transduces an intracellular signal that targets directly or indirectly the inducible promoter regulating expression of the inducible transgene (a) to modify gene expression.

The disclosure further provides a composition comprising the modified, expanded and selected cell population of the methods described herein.

The modified cells of disclosure (e.g., CAR T-cells) can be further modified to enhance their therapeutic potential. Alternatively, or in addition, the modified cells may be further modified to render them less sensitive to immunologic and/or metabolic checkpoints, for example by blocking and/or diluting specific checkpoint signals delivered to the cells (e.g., checkpoint inhibition) naturally, within the tumor immunosuppressive microenvironment.

The modified cells of disclosure (e.g., CAR T-cells) can be further modified to silence or reduce expression of (i) one or more gene(s) encoding receptor(s) of inhibitory checkpoint signals; (ii) one or more gene(s) encoding intracellular proteins involved in checkpoint signaling; (iii) one or more gene(s) encoding a transcription factor that hinders the efficacy of a therapy; (iv) one or more gene(s) encoding a cell death or cell apoptosis receptor; (v) one or more gene(s) encoding a metabolic sensing protein; (vi) one or more gene(s) encoding proteins that that confer sensitivity to a cancer therapy, including a monoclonal antibody; and/or (vii) one or more gene(s) encoding a growth advantage factor. The modified cells may also be modified to silence or reduce expression of the endogenous T cell receptor. Non-limiting examples of genes that may be modified to silence or reduce expression or to repress a function thereof include, but are not limited to genes encoding inhibitory checkpoint signals, intracellular proteins, transcription factors, cell death or cell apoptosis receptors, metabolic sensing protein, proteins that that confer sensitivity to a cancer therapy and growth advantage factors that are disclosed in PCT Publication No. WO 2019/173636, which is incorporated herein by reference in its entirety for examples of genes that may be silenced in the cells of the disclosure.

The modified cells of disclosure (e.g., CAR T-cells) can be further modified to express a modified/chimeric checkpoint receptor. The modified/chimeric checkpoint receptor can comprise a null receptor, decoy receptor or dominant negative receptor. Exemplary null, decoy, or dominant negative intracellular receptors/proteins include, but are not limited to, signaling components downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene. Non-limiting examples of cytokines, cytokine receptors, chemokines and chemokine receptors are disclosed in PCT Publication No. WO 2019/173636, which is incorporated herein by reference in its entirety for examples of modified/chimeric checkpoint receptors that may be expressed in the cells described herein.

Genome modification can comprise introducing a nucleic acid sequence, transgene and/or a genomic editing construct into a cell ex vivo, in vivo, in vitro or in situ to stably integrate a nucleic acid sequence, to transiently integrate a nucleic acid sequence, to produce site-specific integration of a nucleic acid sequence, or to produce a biased integration of a nucleic acid sequence. The nucleic acid sequence can be a transgene.

The stable chromosomal integration can be a random integration, a site-specific integration, or a biased integration. Without wishing to be bound by theory, it is believed that the addition of DNA binding domains to the chimeric transposases and chimeric site-specific transposase fusion proteins described herein improves the site-specificity of the transposases.

The site-specific integration can occur at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that pose a risk to the host organism. Non-limiting examples of potential genomic safe harbors include intronic sequences of the human albumin gene, the adeno-associated virus site 1 (AAVS1), a naturally occurring site of integration of AAV virus on chromosome 19, the site of the chemokine (C—C motif) receptor 5 (CCR5) gene and the site of the human ortholog of the mouse Rosa26 locus.

The site-specific transgene integration can occur at a site that disrupts expression of a target gene. Disruption of target gene expression can occur by site-specific integration at introns, exons, promoters, genetic elements, enhancers, suppressors, start codons, stop codons, and response elements. Non-limiting examples of target genes targeted by site-specific integration include TRAC, TRAB, PD1, any gene encoding an immunosuppressive protein, genes encoding endogenous T cell receptors, and genes encoding proteins involved in allo-rejection.

The site-specific transgene integration can occur at a site that results in enhanced expression of a target gene. Enhancement of target gene expression can occur by site-specific integration at introns, exons, promoters, genetic elements, enhancers, suppressors, start codons, stop codons, and response elements.

The site-specific transgene integration site can be a non-stable chromosomal insertion. The non-stable integration can be a transient non-chromosomal integration, a semi-stable non chromosomal integration, a semi-persistent non-chromosomal insertion, or a non-stable chromosomal insertion. The transient non-chromosomal insertion can be epi-chromosomal or cytoplasmic. In one aspect, the transient non-chromosomal insertion of a transgene does not integrate into a chromosome and the modified genetic material is not replicated during cell division.

The site-specific transgene integration site can be a modified binding site for the DNA targeting domain in a chimeric transposase or a chimeric site-specific transposase fusion protein described herein. For example, the TTAA target DNA integration site for SPB may modified to insert flanking DNA binding sites for the DNA targeting domain comprising three Zinc Finger Motifs (e.g., a DNA targeting domain comprising or consisting of the sequence of SEQ ID NO: 23 or a sequence having at least 75%, at least 80%, at least 85%, at least 90%, at least 95%, at least 98%, or at least 99% identity thereto). For example, it is believed that a DNA targeting domain comprising three Zinc Finger Motifs binds to the DNA sequence GCGTGGGCG. Therefore, the introduction of two copies of the DNA sequence GCGTGGGCG flanking the TTAA target integration site for chimeric site-specific transposase fusion proteins, is believed to improve site-specific integration of the chimeric transposase comprising a DNA targeting domain comprising three Zinc Finger Motifs. In such embodiments, the two copies of the DNA sequence GCGTGGGCG are in reverse (5′) and complement (3′) orientation.

In some embodiments, provided herein is a polynucleotide comprising, in 5′ to 3′ order, the reverse complement of the sequence of a target site for a DNA targeting domain, a first spacer, the TTAA target integration site for SPB, a second spacer, and the sequence of target site for a DNA targeting domain. In some embodiments, the first spacer and the second spacer have the same length. In some embodiments, the first and/or the second spacer are 3 bp in length. In some embodiments, the first and/or the second spacer are 4 bp in length. In some embodiments, the first and/or the second spacer are 5 bp in length. In some embodiments, the first and/or the second spacer are 6 bp in length. In some embodiments, the first and/or the second spacer are 7 bp in length. In some embodiments, the first and/or the second spacer are 8 bp in length. In some embodiments, the first and/or the second spacer are 9 bp in length. In some embodiments, the first and/or the second spacer are 10 bp in length.

The modified target site may be introduced into a cell or a cell line to facilitate targeted genomic engineering. For example, a cell line which has been engineered to comprise a modified target site for a chimeric transposase or a chimeric site-specific transposase fusion protein provided herein can be transfected with said a chimeric transposase or a chimeric site-specific transposase fusion protein as well as a transposon comprising donor DNA such that the donor DNA is inserted at the modified target site. In some embodiments, the cell line is a T cell line. In some embodiments, the ZFM-PB:MosI fusion proteins comprise one amino acid sequences of SEQ ID Nos. 24-27.

The genome modification can be a non-stable chromosomal integration of a transgene. The integrated transgene can become silenced, removed, excised, or further modified.

In some embodiments, the chimeric transposases and chimeric site-specific transposase fusion proteins provided herein have better transposase efficacy than their wildtype equivalents. Transposase activity may be measured by any suitable assay known in the art or described herein, for example, a Split GFP assay. For example, the chimeric transposases and chimeric site-specific transposase fusion proteins provided herein may have comparable on-target genome integration activity to their wildtype counterparts, but have decreased off-target genome integration activity compared to their wildtype counterparts.

In some embodiments, a chimeric PB:MosI transposase comprising a DNA targeting domain provided herein has a ratio of on-target to off-target activity that is increased at least 50-fold, at least about 100-fold, at least about 150-fold, at least about 200-fold, at least about 250-fold, at least about 300-fold, at least about 350-fold, at least about 400-fold, at least about 450-fold, at least about 500-fold, at least about 550-fold, at least about 600-fold, at least about 650-fold, at least about 700-fold, at least about 750-fold, at least about 800-fold, at least about 850-fold, at least about 900-fold, at least about 950-fold, or at least about 1000-fold compared to the wildtype transposase domain (e.g., PB or SPB).

In certain embodiments, the modified cells are used therapeutically in adoptive cell therapy.

Adoptive cell compositions that are “universally” safe for administration to any patient (not just the patient from which they are derived) require a significant reduction or elimination of alloreactivity. Towards this end, cells of the disclosure (e.g., allogenic cells) can be modified to interrupt expression or function of a T-cell Receptor (TCR) and/or a class of Major Histocompatibility Complex (MHC). The TCR mediates graft vs host (GvH) reactions whereas the MHC mediates host vs graft (HvG) reactions. In preferred aspects, any expression and/or function of the TCR is eliminated in a modified cell disclosed herein to prevent T-cell mediated GvH that could cause death to the subject. Thus, in a preferred aspect, the disclosure provides a pure TCR-negative allogeneic T-cell composition (e.g., each cell of the composition expresses the endogenous TCR at a level so low as to either be undetectable or non-existent).

Expression and/or function of MHC class I (MHC-I, specifically, HLA-A, HLA-B, and HLA-C) may be reduced or eliminated to prevent HvG and, consequently, to improve engraftment of cells in a subject. Improved engraftment is believed to results in longer persistence of the cells, and, therefore, a larger therapeutic window for the subject. In some embodiments, expression and/or function of a structural element of MHC-I, Beta-2-Microglobulin (B2M), is reduced or eliminated. Non-limiting examples of guide RNAs (gRNAs) for targeting and deleting MHC activators are disclosed in PCT Application Publication No. WO 2020/051374, which is incorporated herein by reference in its entirety for examples of gRNAs that may be used in the methods described herein.

A detailed description of non-naturally occurring chimeric stimulatory receptors, genetic modifications of endogenous sequences encoding TCR-alpha (TCR-a), TCR-beta (TCR-B), and/or Beta-2-Microglobulin (B2M), and non-naturally occurring polypeptides comprising an HLA class I histocompatibility antigen, alpha chain E (HLA-E) polypeptide is disclosed in PCT Application Publication No. WO 2020/051374, which is incorporated herein by reference in its entirety for examples of non-naturally occurring receptors and polypeptides and other genetic modifications that may be introduced into the cells disclosed herein.

Under normal conditions, full T-cell activation depends on the engagement of the TCR in conjunction with a second signal mediated by one or more co-stimulatory receptors (e.g., CD28, CD2, 4-1BBL) that boost the immune response. However, when the TCR is not present, T cell expansion is severely reduced when stimulated using standard activation/stimulation reagents, including agonist anti-CD3 mAb. Thus, the present disclosure provides a non-naturally occurring chimeric stimulatory receptor (CSR) comprising: (a) an ectodomain comprising a activation component, wherein the activation component is isolated or derived from a first protein; (b) a transmembrane domain; and (c) an endodomain comprising at least one signal transduction domain, wherein the at least one signal transduction domain is isolated or derived from a second protein; wherein the first protein and the second protein are not identical.

The activation component of a CSR described herein can comprise a portion of one or more of a component of a T-cell Receptor (TCR), a component of a TCR complex, a component of a TCR co-receptor, a component of a TCR co-stimulatory protein, a component of a TCR inhibitory protein, a cytokine receptor, and a chemokine receptor to which an agonist of the activation component binds. The activation component can comprise a CD2 extracellular domain or a portion thereof to which an agonist binds.

The signal transduction domain of a CSR described herein can comprise one or more of a component of a human signal transduction domain, a component of a TCR complex, a component of a TCR co-receptor, a component of a TCR co-stimulatory protein, a component of a TCR inhibitory protein, a cytokine receptor, and a chemokine receptor. The signal transduction domain can comprise a CD3 protein or a portion thereof. The CD3 protein can comprise a CD35 domain or a portion thereof.

The endodomain of a CSR described herein can further comprise a cytoplasmic domain. The ectodomain of a CSR can further comprise a signal peptide. The CSR may further comprise a transmembrane domain. The cytoplasmic domain, the signal peptide and/or the transmembrane domain may be derived from the same protein or from different proteins. For example, the signal peptide and the transmembrane domain may be derived from CD8 or CD28 or a combination thereof.

The present disclosure also provides a non-naturally occurring CSR, wherein the ectodomain comprises a modification. The modification can comprise a mutation in or a truncation of the amino acid sequence of the activation component when compared to a wild type sequence of the activation component. The mutation or a truncation of the amino acid sequence of the activation component can comprise a mutation or truncation of a CD2 extracellular domain or a portion thereof to which an agonist binds. The mutation or truncation of the CD2 extracellular domain can reduce or eliminate binding with naturally occurring CD58.

The present disclosure also provides a nucleic acid sequence encoding any CSR disclosed herein. The present disclosure provides a transposon or a vector comprising a nucleic acid sequence encoding any CSR disclosed herein.

The present disclosure provides a cell comprising any CSR disclosed herein. The present disclosure provides a cell comprising a nucleic acid sequence encoding any CSR disclosed herein. The present disclosure provides a cell comprising a vector comprising a nucleic acid sequence encoding any CSR disclosed herein. The present disclosure provides a cell comprising a transposon comprising a nucleic acid sequence encoding any CSR disclosed herein.

The present disclosure provides a composition comprising any CSR disclosed herein. The present disclosure provides a composition comprising a nucleic acid sequence encoding any CSR disclosed herein. The present disclosure provides a composition comprising a vector comprising a nucleic acid sequence encoding any CSR disclosed herein. The present disclosure provides a composition comprising a transposon comprising a nucleic acid sequence encoding any CSR disclosed herein. The present disclosure provides a composition comprising a modified cell disclosed herein or a composition comprising a plurality of modified cells disclosed herein.

Also provided herein are methods for site-specific gene integration. The chimeric transposase and chimeric site-specific transposase fusion proteins provided herein may be used to deliver a transgene to a cell and integrate the transgene into a target site. The target site may be, for example, a genomic safe harbor, i.e., a genomic site where a transgene can be integrated in a manner that ensures that the transgene functions predictably and does not cause alterations of the host genomic DNA sequence. In some embodiments, the target site is a repetitive element, such as a LINE-1 or ALU sequence. Repetitive elements do not encode essential gene products, making it unlikely that that an insertion leads to detrimental changes in the gene expression profile of a cell. There may be one, two or more target sites within one repetitive element. In some embodiments, the target site is located within an intron.

The site-specific integration may be used in vitro or in vivo. An example of an in vivo application is gene therapy, which involves the delivery of a transgene to the genomic DNA of a cell.

### Formulations, Dosages and Modes of Administration

The present disclosure provides formulations, dosages and methods for administration of the compositions and cells described herein. In one aspect, provided herein is a pharmaceutical composition comprising a chimeric transposase or a chimeric site-specific transposase fusion protein described herein and a pharmaceutically acceptable carrier. In another aspect, provided herein is a pharmaceutical composition comprising a modified cell described herein and a pharmaceutically acceptable carrier.

The disclosed compositions and pharmaceutical compositions can comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffinose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN™ 20” and “TWEEN™ 80”), lipids (e.g., phospholipids, fatty acids), steroids (e.g., cholesterol), and chelating agents (e.g., EDTA).

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means. In preferred embodiments, a composition comprising a modified cell described herein is administered intravenously, e.g., by intravenous infusion.

A composition of the disclosure can be prepared for use for parenteral (subcutaneous, intramuscular or intravenous) or any other administration particularly in the form of liquid solutions or suspensions. For parenteral administration, a composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection or infusion can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

It can be desirable to deliver the disclosed compounds to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the disclosed compounds or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

### Methods of Treatment

In another aspect, provided herein are methods of treating a disease or disorder in a subject, the method comprising administering to the subject a composition comprising the modified cells described herein. The terms “subject” and “patient” are used interchangeably herein. In preferred embodiments, the patient is human.

The modified cells may be allogeneic or autologous to the patient. In some preferred embodiments, the modified cell is an allogeneic cell. In some embodiments, the modified cell is an autologous T-cell or a modified autologous CAR T-cell. In some preferred embodiments, the modified cell is an allogeneic T-cell or a modified allogeneic CAR T-cell.

In some embodiments, the disease or disorder treated in accordance with the methods described herein is a cancer. In some embodiments, a method of treatment described herein may delay cancer progression and/or reduce tumor burden.

In some embodiments, the disease or disorder treated in accordance with the methods described herein is an autoimmune disease. In some embodiments, the autoimmune disease is autoimmune neutropenia, Guillain-Barré syndrome, epilepsy, autoimmune encephalitis, Isaacs' syndrome, nevus syndrome, pemphigus vulgaris, deciduous pemphigus, bullous pemphigoid, acquired epidermolysis bullosa, gestational pemphigoid, mucous membrane pemphigoid, antiphospholipid syndrome, autoimmune anemia, myasthenia gravis, autoimmune Graves' disease, thyroid eye disease (TED), Goodpasture syndrome, multiple sclerosis, rheumatoid arthritis, lupus, idiopathic thrombocytopenia purpura (ITP), warm autoimmune hemolytic anemia (WAIHA), chronic inflammatory demyelinating polyneuropathy (CIDP), lupus nephritis, or membranous nephropathy.

The dosage of a pharmaceutical composition to be administered to a subject can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired.

In aspects where the compositions to be administered to a subject in need thereof are modified cells as disclosed herein, between about 1×10³ and about 1×10⁴ cells; between about 1×10⁴ and about 1×10⁵ cells; between about 1×10⁵ and about 1×10⁶ cells; between about 1×10⁶ and about 1×10⁷ cells; between about 1×10⁷ and about 1×10⁸ cells; between about 1×10⁸ and about 1×10⁹ cells; between about 1×10⁹ and about 1×10¹⁰ cells, between about 1×10¹⁰ and about 1×10¹¹ cells, between about 1×10¹¹ and about 1×10¹² cells, between about 1×10¹² and about 1×10¹³ cells, between about 1×10¹³ and about 1×10¹⁴ cells, between about 1×10¹⁴ and about 1×10¹⁵ cells, between about 1×10¹⁵ and about 1×10¹⁶ cells, between about 1×10¹⁶ and about 1×10¹⁷ cells, between about 1×10¹⁷ and about 1×10¹⁸ cells, between about 1×10¹⁸ and about 1×10¹⁹ cells; or between about 1×10¹⁹ and about 1×10²⁰ cells may be administered. In some embodiments, the cells are administered at a dose of between about 5×10⁶ and about 25×10⁶ cells.

In other embodiments, the dosage of cells may depend on the body weight of the person, e.g., between about 1×10³ and about 1×10⁴ cells; between about 1×10⁴ and about 1×10⁵ cells; between about 1×10⁵ and about 1×10⁶ cells; between about 1×10⁶ and about 1×10⁷ cells; between about 1×10⁷ and about 1×10⁸ cells; between about 1×10⁸ and about 1×10⁹ cells; between about 1×10⁹ and about 1×10¹⁰ cells, between about 1×10¹⁰ and about 1×10¹¹ cells, between about 1×10¹¹ and about 1×10¹² cells, between about 1×10¹² and about 1×10¹³ cells, between about 1×1013 and about 1×10¹⁴ cells, between about 1×10¹⁴ and about 1×10¹⁵ cells, between about 1×1015 and about 1×10¹⁶ cells, between about 1×10¹⁶ and about 1×10¹⁷ cells, between about 1×10¹⁷ and about 1×10¹⁸ cells, between about 1×10¹⁸ and about 1×10¹⁹ cells; or between about 1×10¹⁹ and about 1×10²⁰ cells may be administered per kg body weight of the subject.

A more detailed description of pharmaceutically acceptable excipients, formulations, dosages and methods of administration of the disclosed compositions and pharmaceutical compositions is disclosed in PCT Publication No. WO 2019/049816.

The transposon domains and fusion proteins provided herein may be used to deliver a gene therapy. Gene therapy usually involves the delivery of a transgene to the genomic DNA of a cell. Usually, the transgene replaces a gene that is mutated or otherwise not expressed properly in the cell. The fusion proteins, transposase domains, and complexes described herein may be used to deliver a therapeutic transgene to a cell and integrate the transgene into a target site. In some embodiments, a method of treatment comprises introducing into the cell the fusion protein of any one of claims 1-13 and a transposon, wherein the transposon comprises, in 5′ to 3′ order: a 5′ITR, the transgene, and a 3′ ITR.

### Kits

In another aspect, provided herein is a kit comprising a cell line which has been engineered to comprise a modified target site for an SPB or a PBx provided herein within its genome, preferably in a highly expressed genomic region. The kit may further comprise a composition comprising one or more SPB or PBx transposase domains or fusion proteins described herein. In some embodiments, the cell line is a T cell line.

### Terms

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

The term “operatively linked” or its equivalents (e.g., “linked operatively”) means two or more molecules are positioned with respect to each other such that they are capable of interacting to affect a function attributable to one or both molecules or a combination thereof. In the context of nucleic acids, a promoter may be operatively linked to a nucleotide sequence encoding a transpose domain or fusion protein described herein, bringing the expression of the nucleotide sequence under the control of the promoter.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of +2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Table 1, Table 2, and Table 3 below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table 1.

TABLE 1
Conservative Substitutions I

| Side chain characteristics | | Amino Acid |
|---|---|---|
| Aliphatic | Non-polar | G A P I L V F |
| | Polar-uncharged | C S T M N Q |
| | Polar-charged | D E K R |
| Aromatic | | H F W Y |
| Other | | N Q D E |

Alternately, conservative amino acids can be grouped as described in Lehninger, (Biochemistry, Second Edition; Worth Publishers, Inc. NY, N.Y. (1975), pp. 71-77) as set forth in Table 2.

TABLE 2
Conservative Substitutions II

| Side Chain Characteristic | | Amino Acid |
|---|---|---|
| Non-polar | Aliphatic: | A L I V P |
| (hydrophobic) | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table 3.

TABLE 3
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, identity between two sequences may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. In some embodiments, the sequence identify is determined over the entire length of a sequence. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

In certain embodiments, if a sequence has a certain sequence identity (e.g., 75%, 80%, 85%, 90%, 95%, 98%, or 99%) to a certain SEQ ID NO, the sequence and the sequence of the SEQ ID NO have the same length. In certain embodiments, if a sequence has a certain sequence identity (e.g., 75%, 80%, 85%, 90%, 95%, 98%, or 99%) to a certain SEQ ID NO, the sequence and the sequence of the SEQ ID NO only differ due to conservative amino acid substitutions.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### EXAMPLES

The Examples in this section are provided for illustration and are not intended to limit the invention.

### Example 1: Construction of Chimeric PiggyBac (“PB”)-MosI Transposases

Chimeric PB:MosI transposases were created by deleting the CRD domain from PB transposase and replacing it with DNA binding domain(s) of MosI transposase. The PB CRD consists of residues 553-594 of the 594 amino acid PB transposase protein (see SEQ ID NO: 2). The CRD domain was attached to the rest of the transposase through a linker sequence that spans residues 535-552 of the PB transposase sequence. The PB CRD domain may be truncated from the C-terminus of the PB transposase anywhere within the linker sequence. Two fusion points within the linker sequence were selected as exemplary sites. In the first fusion design, the C-terminus of the PB transposase was truncated from residues 545-594 (SEQ ID NO: 3) resulting in the S544 fusion point (S544 PB) to construct the S544 PB-MosI chimera. In the second fusion design, the C-terminus of the PB transposase was truncated from residues 553-594 (SEQ ID NO: 4) resulting in the V552 fusion point (V552 PB) to construct the chimeric V552 PB-MosI transposases.

The C-terminally truncated S544PB and V552 PB sequences were fused to DNA binding domain(s) of MosI. MosI binds with its C-terminus proximal to the transposon ends, unlike PB, which binds with its N-terminus proximal to the transposon ends. The N-terminus of MosI contains two DNA binding domains, which are fused to the catalytic domain of the transposase. The first DNA binding domain spans the first approximately 58 residues of the protein (SEQ ID NO: 8) whereas the two DNA binding domains span the first approximately 111 residues of the protein (SEQ ID NO: 6).

TAL-piggyBac transposase fusion proteins comprising N-terminal deleted piggyBac transposase sequences and integration defective N-terminal piggyBac transposase targeting GFP were produced as described in International Patent Application Publication No. PCT/US2022/77549. Briefly, two pairs of TAL arrays targeting sequences in the GFP coding sequence were designed and each TAL Array containing nine 34 amino acid repeats followed by the 20 amino acid “half” repeat were synthesized flanked by BsmBI type IIS restriction sites. This allowed for cloning of each TAL array in-frame with the rest of the open reading frame with the N-terminal deleted piggyBac transposase sequence in the expression plasmid to generate GFP1R Right TAL-PBx (SEQ ID NO: 40). The PBx transposase sequence was replaced with chimeric PB:MosI transposase sequences to construct chimeric site-specific transposase fusion proteins specifically targeting GFP.

The chimeric PB:MosI transposase sequences were constructed by joining together residues 3-58 of MosI to S544 PBx transposase using a GGGGS linker sequence (SEQ ID NO: 86) and inserted in place of PBx transposase sequence to create GFP1R TAL-PB: S544-MosI-58 (SEQ ID NO: 41). Similarly, residues 3-58 of MosI were fused to V552 PBx transposase sequence using a GGGGS linker (SEQ ID NO: 86) and inserted in place of the PBx transposase sequence to create GFP1R TAL-PB: V552-MosI-58 (SEQ ID NO: 42). Also, residues 3-111 of MosI were fused to S544 PB using a GGGGS linker (SEQ ID NO: 86) and inserted in place of the PBx transposase sequence to create GFP1R TAL-PB: S544-MosI-111 (SEQ ID NO: 43). Finally, residues 3-111 of MosI were fused to PiggyBac at V552 PBx using a GGGGS linker (SEQ ID NO: 86) and inserted in place of the PBx transposase sequence to create GFP1R TAL-ssSPB: V552-MosI-111 (SEQ ID NO: 44).

### Example 2: Construction of Chimeric PB:MosI Inverted Terminal Repeats (ITRs)

This example illustrates exemplary methods for constructing chimeric PB:MosI ITR sequences.

The chimeric PB:MosI transposases prepared in Example 1 each comprise the PB DNA binding and dimerization domain (DDBD) fused to the DNA binding domain(s) from MosI. Thus, chimeric PB:MosI ITR sequences comprising the binding site for PB DDBD fused to the binding site for MosI DNA binding domains were constructed.

The 35 bp LE PB ITR sequence (SEQ ID NO: 12) was truncated after position 16, deleting the 19 bp CRD binding site. To this end, a 23 bp MosI sequence (SEQ ID NO: 13) containing the binding sites for the two MosI DNA binding domains was fused in reverse orientation to create the LE PB:MosI ITR (SEQ ID NO: 15). In addition, alternative versions of the chimeric ITR comprising various spacer lengths between the PB and MosI ITR domains in the chimeric protein were designed to test the effect of linker length. In one instance, one bp of the MosI binding site was deleted adjacent to the DDBD binding site to create LE PB:MosI ITR-1 (SEQ ID NO: 16). Additionally, one or two bp of DNA was added between the MosI binding site and the DDBD binding site to create LE PB:MosI ITR+1 (SEQ ID NO: 17) and LE PB:MosI ITR+2 (SEQ ID NO: 18). The 63 bp RE ITR (SEQ ID NO: 65) was similarly modified to create RE PB:MosI ITR (SEQ ID NO: 19), RE PB:MosI ITR-1 (SEQ ID NO: 20), RE PB:MosI ITR+1 (SEQ ID NO: 21), RE PB:MosI ITR+2 (SEQ ID NO: 22).

The chimeric PB:MosI transposases and the chimeric ITR sequences were analyzed for their relative excision activity.

### Example 3: Methods for Measuring Excision Activity of Chimeric PB-MosI Transposases Using Chimeric PB:MosI ITRs

This example describes an assay designed to measure the excision activity of chimeric PB-MosI transposases and chimeric ITRs. In this assay, each of the chimeric PB-MosI transposases prepared in Example 1 was co-administered to cells together with a reporter transposon construct, in which the transposon comprises a DNA nucleotide sequence encoding a non-functional GFP in which the coding sequence has been disrupted by an intervening piece of DNA flanked by TTAA sequences and one set of the inverted terminal repeat (ITR) sequences prepared in Example 2. The TTAA sequences and ITRs serve as recognition sites for the chimeric PB:MosI transposase and if the chimeric transposase possesses excision activity, the intervening DNA will be excised, restoring the intact, full-length coding sequence of the GFP gene. Thus, the chimeric transposases possessing transposase activity for constructs comprising the cognate chimeric ITR sequence produce GFP positive cells in this assay that may be identified and quantified by FACS.

Briefly, the GFP reporter system comprises an EF1a promoter (SEQ ID NO: 45) driving expression of a GFP reporter (SEQ ID NO: 46) followed by a SV40 polyadenylation sequence (SEQ ID NO: 47). The GFP reporter is disrupted by a transposon at a TTAA sequence, breaking it into the first GFP section (SEQ ID NO: 48) and the second GFP section (SEQ ID NO: 49). Disrupted GFP reporters for each chimeric ITR from Example 2 were constructed harboring a PB:MosI ITR transposon (SEQ ID NO: 50), a PiggyBac: MosI ITR-1 transposon (SEQ ID NO: 51), a PiggyBac: MosI ITR+1 transposon (SEQ ID NO: 52), or a PiggyBac: MosI ITR+2 transposon (SEQ ID NO: 53).

Using the GFP reporter system, the excision activity of GFPR1 TAL-PB: S544-MosI-58, GFP1R TAL-PB: V552-MosI-58, GFPR1 TAL-PB: S544-MosI-111, and GFP1R TAL-ssSPB: V552-MosI-111 transposases described in Example 1 was determined using each of the chimeric PB:MosI ITRs prepared in Example 2. On Day 0, each reporter was co-transfected into HEK293T cells with each chimeric transposase. Briefly 120,000 cells were plated in 24 well plates in 500 μL of DMEM medium supplemented with 10% v/v FBS one day before transfection. On Day 1, 50 ng of the transposase expressing vector was combined with 450 ng of the reporter and transfected using 1 μL of JetPrime transfection reagent (Polyplus Transfection™) in accordance with the manufacturer's instructions. One day after transfection, the percentage of GFP positive cells was measured by flow cytometry. The results are shown Table 4.

TABLE 4

| | Percentage GFP Positive Cells (Transposon Excision) | | | | | | | | | | | |
|---|---|---|---|---|---|---|---|---|---|---|---|---|
| | PB:MosI ITR-1 | | | PB:MosI ITR | | | PB:MosI ITR + 1 | | | PB:MosI ITR + 2 | | |
| | Rep1 | Rep2 | Avg | Rep1 | Rep2 | Avg | Rep1 | Rep2 | Avg | Rep1 | Rep2 | Avg |
| S544- | 1.40 | 1.44 | 1.42 | 7.71 | 6.98 | 7.35 | 2.84 | 2.78 | 2.81 | 2.82 | 2.85 | 2.84 |
| MosI-58 | | | | | | | | | | | | |
| S544- | 1.07 | 0.84 | 0.96 | 6.44 | 6.64 | 6.54 | 2.75 | 2.36 | 2.56 | 2.42 | 2.75 | 2.59 |
| MosI-111 | | | | | | | | | | | | |
| V552- | 0.96 | 0.77 | 0.87 | 4.92 | 4.87 | 4.90 | 2.34 | 2.83 | 2.59 | 4.00 | 3.76 | 3.88 |
| MosI-58 | | | | | | | | | | | | |
| V552- | 0.86 | 0.69 | 0.78 | 4.38 | 4.35 | 4.37 | 3.23 | 3.10 | 3.17 | 2.49 | 2.65 | 2.57 |
| MosI-111 | | | | | | | | | | | | |
| No | 0.16 | 0.08 | 0.12 | 0.06 | 0.08 | 0.07 | 0.13 | 0.13 | 0.13 | 0.05 | 0.07 | 0.06 |
| Transposase | | | | | | | | | | | | |

As shown in Table 4, chimeric transposases with MosI inserted at the S544 fusion point resulted in higher transposon excision activity than those with MosI inserted at the V552 fusion point. Additionally, higher transposon excision activity was observed with the reporters containing the PB:MosI ITR rather than the ITRs having −1, +1, or +2 spacing.

The chimeric ITRs tested in Table 4 contain a 23 bp sequence comprising the binding sites for both MosI DNA binding domains. Since the first 58 residues of MosI contain a single DNA binding domain, a series of truncated ITR sequences were constructed in which the MosI portion of the ITR comprises a binding site for just the single domain.

In a second experiment, the MosI binding site was shortened from 23 bp to 14 bp to create left end LE PB:MosI ITR short (SEQ ID NO: 54) and right end RE PB:MosI ITR short (SEQ ID NO: 55). These shorter ITRs were used to create a disrupted GFP reporter as described in Example 1 using PB:MosI ITR short transposon (SEQ ID NO: 56). The short and original chimeric ITR reporters were tested along with the S544 fusion point chimeric transposases, S544-MosI-58 and S544-MosI-111 using the GFP Reporter system above. The results are shown in Table 5.

TABLE 5

| | Percentage GFP Positive Cells (Transposon Excision) | | | | | |
|---|---|---|---|---|---|---|
| | Piggy Bac:MosI ITR | | | Piggy Bac:MosI ITR short | | |
| | Rep1 | Rep2 | Avg | Rep1 | Rep2 | Avg |
| S544-MosI-58 | 3.57 | 4.10 | 3.84 | 2.08 | 2.29 | 2.19 |
| S544-MosI-111 | 3.97 | 3.80 | 3.89 | 2.36 | 1.81 | 2.09 |
| No Transposase | 0.07 | 0.05 | 0.06 | 0.08 | 0.03 | 0.05 |

As shown in Table 5, the original and short ITRs each resulted in functional transposon excision activity; however, the original 23 bp version resulted in higher activity for chimeric transposases comprising either the residue 3-58 or residue 3-111 MosI fusions as observed by the percentage of GFP positive cells.

### Example 4: Construction and Analysis of Chimeric TAL-PB:MosI Transposases Designed for Site-specific Transposition of a Small Transposon in LINE-1 Elements

This Example illustrates the construction of chimeric TAL-PB:MosI transposase fusion protein compositions that are useful in methods for achieving site-specific transposition at a specific target locus, LINE1.

LINE1-specific TAL DNA binding domains previously were joined to N-terminal deleted piggyBac transposase sequences to construct LINE1 TAL-ssSPB fusion proteins (e.g., left; SEQ ID NO: 57; right SEQ ID NO: 58). Similar to the construction of chimeric TAL-PB:MosI transposase fusion proteins targeting GFP, the LINE1 left and right TAL-ssSPB fusion proteins were converted to chimeric site-specific PB:MosI transposases by replacing the PB transposase sequence with a PB:MosI transposase as described above in Example 1. Two versions of the left LINE1 L2 TAL-PB:MosI transposase were created using the S544 fusion point and either the 3-58 residue MosI fragment (SEQ ID NO: 59) or the 3-111 residue MosI fragment (SEQ ID NO: 60). Likewise, two versions of the right LINE1 R2.2 TAL-PB:MosI transposases were created using the S544 fusion point and either the 3-58 residue MosI fragment (SEQ ID NO: 61) or the 3-111 residue MosI fragment (SEQ ID NO: 62). A small 365 bp transposon comprising PB minimal ITRs (SEQ ID NO: 63) and a small 353 bp transposon comprising chimeric PB:MosI (23 bp) ITRs (SEQ ID NO: 50) from Example 2 were cloned into a 4.5 kb donor vector. On Day 0, 450 ng of each transposon donor was co-transfected into 120,000 HEK293T cells (plated one day earlier) along with 50 total ng of the pair of LINE1 TAL-PB pair using 1 μL of JetPrime transfection reagent (Polyplus Transfection™) in accordance with the manufacturer's instructions. On Day 3, genomic DNA was harvested from transposed cells and site-specific integration of the transposon into the target sites in the forward orientations was quantified by digital droplet PCR (ddPCR) to determine the number of integrations per haploid genome. The results are shown in Table 6.

TABLE 6

| | Integrations per haploid genome | | | | | |
|---|---|---|---|---|---|---|
| | PB:MosI ITR | | | PB Minimal ITR | | |
| | Rep1 | Rep2 | Avg | Rep1 | Rep2 | Avg |
| TAL-PB-S544-MosI-58 | 1.66 | 1.71 | 1.69 | 0.95 | 1.00 | 0.98 |
| TAL-PB-S544-MosI-111 | 1.99 | 1.94 | 1.97 | 0.74 | 0.74 | 0.74 |
| TAL-ssSPB | 1.03 | 1.07 | 1.05 | 2.91 | 2.40 | 2.66 |

As shown in Table 6, the chimeric MosI 3-111 residue version of the chimeric site-specific PB:MosI transposases resulted in higher editing rates than the chimeric MosI 3-58 residue site-specific PB:MosI version. The two chimeric transposases each catalyzed higher site-specific transposition of the transposon using the chimeric PB:MosI ITRs than of the transposon comprising the non-chimeric PB minimal ITRs. The non-chimeric TAL-ssSPB transposase catalyzed higher site-specific transposition of the transposon using the non-chimeric PB minimal ITRs than of the transposon comprising the chimeric PB:MosI ITRs. These data suggest the chimeric and the non-chimeric transposases can be used as orthogonal site-specific transposition systems to simultaneously deliver transposons to two unique genetic loci, one site targeted with a chimeric PB:MosI ITR target and the other site targeted with a wild type PB minimal ITR.

### Example 5: Site-specific Transposition of Large Cargo Transposons at a Specific Genetic Locus Using Chimeric PB:MosI Transposases

This Example illustrates site-specific transposition at a specific target locus, LINE-1, of transposons comprising a large DNA cargo.

The chimeric TAL-PB:MosI transposases constructed in Example 4 and control transposase TAL-ssSPB were analyzed for their ability to site specifically transpose a transposon comprising a large DNA cargo into LINE1 elements.

The transposon donor nanoplasmid comprising a PB transposon containing from 5′ to 3′ direction: a first TTAA sequence, a 309 bp fragment containing the PB 5′ ITR and part of the UTR, a “large cargo” consisting of an EF1a promoter, (SEQ ID NO: 45) a puromycin resistance gene, a 2A peptide, and a GFP reporter, followed by a 238 bp fragment containing the PB 3′ ITR and part of the UTR, and a second TTAA sequence (SEQ ID NO: 64). This donor plasmid was used to analyze wild type PB control TAL-ssSPB and a negative control PB transposase that comprises mutations rendering the transposase integration defective.

For analysis of chimeric TAL-PB:MosI transposases, the transposon donor nanoplasmid was modified to replace the 309 bp LE ITR/UTR and 238 bp RE ITR/UTR with the chimeric PB:MosI (23 bp) ITRs of SEQ ID NO: 1 and, SEQ ID NO: 19). The results are shown in Table 7.

TABLE 7

Integrations per
haploid genome

| | PB:MosI | PB Full |
|---|---|---|
| | ITR | ITR |
| TAL-PB:MosI-S544-MosI-58 | 0.87 | 0.31 |
| TAL-PB:MosI-S544-MosI-111 | 1.76 | 0.33 |
| TAL-ssSPB | 0.36 | 0.95 |
| PBx (Integration defective) | 0.02 | 0.01 |

As shown in Table 7, the two chimeric and non-chimeric transposases each functioned as orthogonal systems showing a high degree of specificity for their respective ITRs. The chimeric TAL-PB:MosI 3-111 residue version of the chimeric PB:MosI transposase resulted in higher editing rates than the chimeric TAL-PB:MosI 3-58 residue version as well as the non-chimeric version, TAL-ssSPB. As a negative control, integration defective PBx transposase comprising no TAL DNA targeting domains resulted in only background integration levels.

### Example 6: Construction of Chimeric PB:MosI Transposases Comprising PB Hyperactive Mutations (“SPB”) that Increase Transposition Frequency

This example illustrates methods for constructing chimeric SPB:MosI transposases and analysis of integration and excision activity. In this example, SPB with an N-terminal NLS (SEQ ID NO: 5) was modified as described above to create SPB:MosI chimeric transposases.

More specifically, residues 3-58 of MosI (SEQ ID NO: 8) were fused to SPB at S544 to create SPB:MosI-S544-58 (SEQ ID NO: 9) and residues 3-111 of MosI (SEQ ID NO: 6) were fused to SPB at S544 to create SPB:MosI-S544-111 (SEQ ID NO: 7). The chimeric SPB:MosI transposases were tested using disrupted GFP Reporter system harboring the PB:MosI ITR transposon (SEQ ID NO: 50), the PB:MosI ITR short transposon (SEQ ID NO: 56), or the PB minimal ITR transposon (SEQ ID NO: 63). On Day 0, each reporter construct was co-transfected into HEK293T cells with each chimeric PB:MosI transposase. Briefly 120,000 cells were plated in 24 well plates in 500 μL of DMEM supplemented with 10% v/v FBS one day before transfection. On Day 0, 10 ng of the transposase expressing vector was combined with 490 ng of the reporter and transfected using 1 μL of JetPrime transfection reagent (PolyPlus Transfection™). On Day 1, the percentage of GFP positive cells was measured by flow cytometry. The results are shown in Table 8.

TABLE 8

| | Percentage GFP Positive Cells (Transposon Excision) | | | | | | | | | | | |
|---|---|---|---|---|---|---|---|---|---|---|---|---|
| | PB:MosI ITR | | | | PB:MosI ITR short | | | | PB Minimal ITR | | | |
| | Rep1 | Rep2 | Rep3 | Avg | Rep1 | Rep2 | Rep3 | Avg | Rep1 | Rep2 | Rep3 | Avg |
| SPB:MosI- | 19.2 | 20.0 | 18.5 | 19.2 | 21.8 | 22.7 | 24.3 | 22.9 | 1.4 | 1.0 | 1.0 | 1.1 |
| S544-58 | | | | | | | | | | | | |
| SPB:MosI- | 17.0 | 17.0 | 16.7 | 16.9 | 7.2 | 7.8 | 7.5 | 7.5 | 0.1 | 0.1 | 0.1 | 0.1 |
| S544-111 | | | | | | | | | | | | |
| SPB | 0.1 | 0.2 | 0.3 | 0.2 | 0.3 | 0.4 | 0.5 | 0.4 | 52.4 | 49.2 | 50.8 | 50.8 |

As shown in Table 8, the chimeric SPB:MosI transposases catalyzed excision of the transposon, as measured by percentage of GFP positive cells, for transposons comprising chimeric PB:MosI ITRs but not for transposons comprising the non-chimeric minimal PB ITRs. The non-chimeric SPB transposase catalyzed excision of the transposon for the non-chimeric minimal PB ITRs but not for transposons with the chimeric PB:MosI ITRs demonstrating the specificity of the chimeric PB:MosI transposase/PB:MosI ITR transposon combination.

What is claimed is:

1. A chimeric transposase comprising, in N-terminal to C-terminal order: (i) a target-specific DNA binding domain, (ii) a truncated Super piggyBac (SPB) transposase comprising a C-terminal Cysteine Rich Domain (CRD) deletion within amino acid residues 535-594 of the sequence of the SPB comprising the sequence set forth in SEQ ID NO: 1, and (iii) one or more MosI DNA binding domain(s).

2. The chimeric transposase of claim 1, wherein the truncated SPB transposase of (ii) comprises the sequence set forth in any one of SEQ ID NOs.: 68-84.

3. The chimeric transposase of claim 1 or 2, wherein the truncated PB transposase comprises one or more hyperactive mutation selected from I30V, S103P, G165S, M226F, M282V, S509G, N538K or N571S of SEQ ID NO: 1.

4. The chimeric transposase of any one of claims 1-3, wherein the PB transposase comprising the CRD deletion further comprises an in-frame N-terminal nuclear localization sequence (NLS) comprising the amino acid sequence of SEQ ID NO: 39.

5. The chimeric transposase of any one of claims 1-4, wherein the chimeric transposase comprises two MosI DNA binding domains comprising the amino acid sequence set forth in SEQ ID NO: 6.

6. The chimeric transposase of any one of claims 1-4, wherein the chimeric transposase comprises one MosI DNA binding domain comprising the amino acid sequence set forth in SEQ ID NO: 8.

7. The chimeric transposon of any one of claims 1-6, wherein the target-specific DNA binding domain is a zing finger domain or a TAL array.

8. A polynucleotide comprising a nucleic acid sequence encoding the chimeric transposase of any one of claims 1-7.

9. A vector comprising the polynucleotide of claim 8.

10. A chimeric transposon inverted terminal repeat (ITR) polynucleotide, comprising in the 5′ to 3′ direction: (i) a polynucleotide comprising nucleotides 1-16 of the nucleic acid sequence set forth in SEQ ID NO: 12 and (ii) a polynucleotide comprising two MosI DNA binding sites.

11. The chimeric transposon ITR polynucleotide of claim 10, wherein the polynucleotide comprising two MosI DNA binding sites comprises the nucleic acid sequences of SEQ ID NOs: 13 or 14.

12. The chimeric transposon ITR polynucleotide of claim 10 or 11, wherein the chimeric transposon ITR polynucleotide comprises the nucleic acid sequence of SEQ ID NOs: 15 or 16.

13. The chimeric transposon ITR polynucleotide of any one of claims 10-12, wherein the chimeric transposon ITR polynucleotide comprises the nucleic acid sequence of SEQ ID NOS: 17 or 18.

14. A vector comprising the chimeric transposon ITR polynucleotide of any one of claims 10-13.

15. A chimeric transposon ITR polynucleotide, comprising in the 5′ to 3′ direction: (i) a polynucleotide comprising nucleotides 1-16 of the nucleic acid sequence set forth in SEQ ID NO: 65 and (ii) a polynucleotide comprising two MosI DNA binding sites.

16. The chimeric transposon ITR polynucleotide of claim 15, wherein the polynucleotide comprising two MosI DNA binding sites comprises the nucleic acid sequence of SEQ ID NOs: 13 or 14.

17. The chimeric transposon ITR polynucleotide of claim 15, wherein the chimeric transposon ITR polynucleotide comprises the nucleic acid sequence of any one of SEQ ID NOs: 19-22.

18. A vector comprising the chimeric transposon ITR polynucleotide of any one of claims 15-17.

19. A LINE1-targeting chimeric site-specific transposase fusion protein comprising, in the N-terminal to C-terminal direction, (i) a left or a right LINE1 TAL-targeting DNA binding domain, (ii) a linker sequence, and (iii) a chimeric PB:MosI transposase.

20. The LINE1-targeting chimeric site-specific transposase fusion protein of claim 19, wherein the chimeric PB:MosI transposase comprises the amino acid sequence set forth in SEQ ID NO: 9 or 7.

21. The LINE1-targeting chimeric site-specific transposase fusion protein of claim 20, wherein the left or the right LINE1 TAL-targeting DNA binding domain comprises the amino acid sequence set forth in SEQ ID NOs: 66 or 67.

22. A transposon, comprising (i) a chimeric LE PB:MosI ITR polynucleotide and (ii) a chimeric RE PB:MosI ITR polynucleotide, wherein the LE PB:MosI ITR polynucleotide and an RE PB:MosI ITR polynucleotide comprise the same nucleic acid sequence.

23. The transposon of claim 22, wherein the chimeric LE PB:MosI ITR polynucleotide and the chimeric RE PB:MosI ITR polynucleotide each comprise the nucleic acid sequence of any one of SEQ ID NOs: 15-22.

24. A transposon, comprising (i) a chimeric LE PB:MosI ITR polynucleotide comprising the nucleic acid sequence of SEQ ID NO: 54 and (ii) a chimeric RE PB:MosI ITR polynucleotide comprising the nucleic acid sequence of SEQ ID NO: 55.

25. A transposon comprising the nucleic acid sequence of SEQ ID NO: 56.

26. A method of integrating a transgene into a genomic target site of a cell, the method comprising introducing into the cell the chimeric transposase of any one of claims 1-7 and a transposon, wherein the transposon comprises, in 5′ to 3′ order: a 5′ITR, the transgene, and a 3′ ITR, wherein the 5′ITR is a chimeric transposon inverted terminal repeat (ITR) of any one of claims 10-13 and the 3′UTR is a chimeric transposon inverted terminal repeat (ITR) of any one of claims 15-17.

27. The method of claim 26, wherein the 5′ITR chimeric transposon inverted terminal repeat comprises the nucleic acid sequence set forth in SEQ ID NO: 15 and/or wherein the 3′ITR chimeric transposon inverted terminal repeat comprises the nucleic acid sequence set forth in SEQ ID NO: 21.

28. The method of any one of claim 26 or 27, wherein the genomic target site is located in a repetitive element.

29. The method of claim 28, wherein the repetitive element is a LINE element.

30. A method for site-specific transposition of a DNA molecule into the genome of a cell, comprising introducing into the cell:
a) a nucleic acid encoding a chimeric site-specific transposase fusion protein comprising a DNA binding domain and a chimeric transposase comprising, in N-terminal to C-terminal order: a piggyBac transposase comprising a C-terminal Cysteine Rich Domain (CRD) deletion and one or more MosI DNA binding domain(s);
b) wherein the fusion protein is expressed in the cell; and
c) a DNA molecule comprising a transposon comprising a chimeric LE transposon ITR polynucleotide and a chimeric RE transposon ITR polynucleotide; wherein the expressed chimeric site-specific transposase fusion protein integrates the transposon by site-specific transposition into the TTAA sequence of cellular genome.

31. A method for generating an engineered cell by site-specific transposition, comprising introducing into the cell:
a) a nucleic acid encoding a chimeric site-specific transposase fusion protein comprising a DNA binding domain and a chimeric transposase comprising, in N-terminal to C-terminal order: a piggyBac transposase comprising a C-terminal Cysteine Rich Domain (CRD) deletion and one or more MosI DNA binding domain(s);
b) wherein the chimeric site-specific transposase fusion protein is expressed in the cell; and
c) a DNA molecule comprising a transposon comprising a chimeric LE transposon ITR polynucleotide and a chimeric RE transposon ITR polynucleotide; wherein the expressed chimeric site-specific transposase fusion protein integrates the transposon by site-specific transposition into the TTAA sequence into the genome of the cell thereby generating the engineered cell.
